(19) United States
(12) Patent Application Publication
(10) Pub. No.: US 2021/0162398 A1
(43) Pub. Date: Jun. 3, 2021

(54) MEDICAL TESTING DEVICE

(71) Applicant: RPI Consulting, LLC, Encino, CA (US)

(72) Inventor: Ozlem Equils, Encino, CA (US)

(73) Assignee: RPI Consulting, LLC, Encino, CA (US)

(21) Appl. No.: 17/106,099

(22) Filed: Nov. 28, 2020

Related U.S. Application Data
(60) Provisional application No. 62/943,180, filed on Dec. 3, 2019.
(60) Provisional application No. 62/977,054, filed on Feb. 14, 2020.

(51) Int. Cl. B01L 3/02; G01N 15/14; G01N 33/543; C12Q 1/6806; C12Q 1/686; B01L 9/00

(52) U.S. Cl. CPC B01L 3/0217; G01N 15/14; G01N 33/54326; C12Q 1/6806; B01L 2200/0605; B01L 9/543; B01L 2300/0829; B01L 2400/043; B01L 2400/0478; C12Q 1/686

(57) ABSTRACT

A medical testing device is disclosed herein. The robotic unit of the medical testing device contains a hand and arm for handling a plurality of biological samples. The hand can move the plurality of biological samples in the x, y, z directions and rotate 360 degrees. The medical testing herein allows for uniform testing of the plurality of biological samples.

### CROSS-REFERENCE TO RELATED APPLICATIONS

This application claims the benefit of U.S. Provisional Application No. 62/943,180, filed Dec. 3, 2019, and U.S. Provisional Application 62/977,054, filed Feb. 14, 2020, both of which are incorporated by reference herein in their entirety.

### BACKGROUND OF THE INVENTION

### Field of the Invention

This invention relates to systems and methods for monitoring and determining functional immune responses of an organism by measuring the level of inflammatory mediators such as cytokines, chemokines, leukotrienes, etc.

### Description of the Related Art

Immune system activation plays a role in many diseases such as atherosclerosis, chronic lung disease, inflammatory bowel disease, psoriasis etc. Cytokines and/or chemokines play a role in the pathogenesis of a specific disease and are produced by peripheral blood mononuclear cells (PBMC) or a subset of PBMC or whole blood in response to ex vivo stimulation. The stimulants may be microbial antigens, PHA, HLA, toxins, chemicals, and so forth that play a role in the pathogenesis of those diseases. Measurements of these mediators can determine the appropriateness of immune system function and immune system responses of a patient. The readout is the level of inflammatory mediator production such as cytokine, chemokine, leukotriene, or acute phase reactant and so forth. The assays may investigate and assess the whole immune system function of the patient (pro-inflammatory and anti-inflammatory) in the context of a susceptible organ system such as heart, joints, intestines, brain, uterus, skin etc.

Interleukin 1 (IL1) plays a role in successful maternal-blastocyst dialogue and implantation of the embryo, placentation and ultimately successful pregnancy outcome, which is full term delivery with no maternal or fetal complications.

IL1 also mediates healthy functioning of the female gynecologic system such as menstruation and menopause.

Ex vivo IL-1 protein expression and levels of IL-1 mediators, such as IL1 Receptor antagonist (IL-1Ra) level, may be monitored to screen, treat, and track the progress of female obstetrics and gynecologic conditions and treatments where IL-1Ra is administered in subjects/patients. Subjects/patients may be nonpregnant, pregnant or undergoing treatments with assistive reproductive technology (ART), or going through menopause or post menopause.

Additionally, patients/subjects' IL1 system responses may be screened to identify those at risk for poor placentation or poor pregnancy outcome, such as recurrent pregnancy loss, abruptio placenta, pre-eclampsia, HELLP syndrome, preterm delivery, gestational diabetes or post-partum depression. Clinical subjects/patients that may benefit from these screenings include humans, cattle, sheep, rats, mice, and other animal organisms.

Further, the IL-1 system is at the center of inflammatory, apoptotic, infectious, endocrine and vascular molecular pathways that lead to the pathogenesis of endometriosis. Endometriosis is a frequent gynecological disease affecting up to 5-10% of the women of reproductive age and a major cause of abdominal pain, dysmenorrhea, dyspareunia. It is one of the top three causes of infertility. The etiology of endometriosis appears to be multifactorial and involve a complex interplay of multiple genetic, environmental, hormonal, and immunological factors. Escherichia coli lipopolysaccharide (LPS) has been found in the menstrual blood and peritoneal fluid of women with endometriosis and retrograde exposure to microbial antigens may also activate the inflammatory pathways and promote the growth of endometrial tissue originating from retrograde menstruation.

There is 5-10 years of delay in the diagnosis of endometriosis. The current gold standard for diagnosis of endometriosis is laparoscopic and histologic proof of the lesions outside of the uterus. However, laparoscopy is invasive and expensive. Although rare, laparoscopy is associated with potential surgical complications. In 10-15% of the cases, laparoscopy is falsely negative.

Physical examination is useful in the diagnosis of endometriosis but bimanual examination may not be feasible for non-sexually active adolescents/young adults and may not identify early-stage, superficial disease.

Although imaging with ultrasound or MR′ may improve the sensitivity of physical examination, not all endometriosis lesions can be visualized by imaging. Imaging is expensive and not easily accessible to all women globally.

Peripheral blood may be examined for marker(s) of endometriosis; however, no diagnostic marker(s) from unprocessed blood has been identified and validated so far.

Animal models and genetic and epidemiologic studies establish that the IL1 system plays a key role in the pathogenesis of endometriosis. However, measurement of serum levels of IL1 system components and polymorphism analyses have not been shown to be useful for the diagnosis of endometriosis.

### BRIEF SUMMARY OF THE INVENTION

Provided is a medical testing device comprising:
a base component comprising a door unit and a plurality of chamber units;
a plurality of walls operatively connected to the base component;
a robotic unit operatively connected to at least one wall of the plurality of walls and a volume transfer unit;
an assay section operatively connected to at least one chamber unit of the plurality of chamber units;
a computer unit operatively connected to the base component; and
a plurality of instruments for measuring characteristics of samples handled by the robotic unit.

Also provided is a method for measuring levels of proteins using a medical device acting on specialized tubes, wherein the medical device comprises:
a base component comprising a door unit and a plurality of chamber units;
a plurality of walls operatively connected to the base component;
a robotic unit operatively connected to at least one wall of the plurality of walls and a first volume transfer unit;
an assay section operatively connected to at least one chamber unit of the plurality of chamber units;
a computer unit operatively connected to the base component;
a plurality of instruments for measuring characteristics of samples handled by the robotic unit, wherein the samples contain the proteins; and
wherein the robotic unit moves in a first direction and second direction for uniform handling of at least two samples contained within the specialized tubes.

### BRIEF DESCRIPTION OF THE SEVERAL VIEWS OF THE DRAWINGS

Those of skill in the art will understand that the drawings, described below, are for illustrative purposes only. The drawings are not intended to limit the scope of the present teachings in any way.

FIG. 1 and FIG. 2 are plots of IL-1Ra responses.

FIG. 3 and FIG. 4 are histograms for induced IL-1Ra.

FIG. 5 is a depiction of an assembly where a medical testing device is connected to an instrument and computer.

FIG. 6 is a depiction of components for assembling a base of the medical testing device to wall units and a control unit.

FIG. 7 is a depiction of components for assembling a door and top cover to the base of the medical testing device.

FIG. 8 is a depiction of components for assembling a first robotic transfer unit to the control unit.

FIG. 9 is a depiction of the first robotic transfer unit transferring volume(s) of samples.

FIG. 10 is a depiction of a path for moving a well or test tube rack from a chamber in the base of the medical testing device to an instrument.

FIG. 11 is a depiction of assembling a second robotic transfer unit.

FIG. 12 is a depiction of moving the well or test tube rack from the chamber in the base of the medical testing device to the instrument via the second robotic transfer unit.

FIG. 13 is a depiction of a stepwise path for moving the well or test tube rack from the chamber in the base of the medical testing device to the instrument via the second robotic transfer unit.

FIG. 14 is a depiction of the first robotic transfer unit transferring volumes of samples for diagnosis, disposing a tip to be replaced with a new tip.

FIG. 15 is a depiction of the first robotic transfer unit in combination with a n-well plate equipped with a magnetic strips.

FIG. 16 is a depiction of the first robotic transfer unit in combination with a n-well plate equipped with magnetic beads.

FIGS. 17-20 are depictions of the transfer of volume of samples and the disposal of tips at different times.

FIG. 21 is a depiction of protein responses in instances of endometriosis.

FIG. 22 is a depiction of specialized tubes for assessing endometriosis.

FIG. 23 is a depiction of using specialized tubes and the medical devices herein for uniform analysis of biological samples.

### DETAILED DESCRIPTION OF THE INVENTION

The present invention provides a medical testing device for monitoring and determining expression levels of proteins, such as cytokine or chemokines, and their receptor antagonist levels produced by peripheral blood mononuclear cells (PBMC), whole blood or another subset of PBMC.

The medical testing device herein can measure IL1 system responses, which may be used for diagnostic purposes. Since women with obstetrics and gynecologic conditions are otherwise healthy, we hypothesized that only the measurement of ex vivo immune stimulation of the PBMC or whole blood would reveal those women who have dysfunctional immune responses. After using the medical testing device for preterm delivery diagnosis, we observed statistically significant differences in IL-1 system responses (IL-1Ra levels) of PBMC from women with history of preterm delivery in comparison to women with history of full term delivery.

Whole blood may be used in the medical testing device herein. The medical testing devices herein may be portable, disassembled, and rearranged to support analytical biochemistry techniques. The analytical biochemistry techniques facilitated by the medical testing devices include, but are not limited to: enzyme-linked immunosorbent assays (ELISAs); flow cytometry; polymerase chain reactions (PCR);

photometry/spectrophotometry; protein interactions (e.g., DNase footprinting assay, filter binding assay, and get shift assay); a secretion assay for identifying cells secreting a protein (e.g., cytokine); and colorimetric protein assays (e.g., bicinchoninic acid assay, Multiples Luminex assay).

The medical testing devices herein may provide the advantages of: (i) reducing preparation time for biological/medical assays; (ii) standardizing testing of samples; (iii) parallelizing testing of samples; (iv) reducing error-prone techniques when performing biological/medical assays, such as changing pipette or other volume transferring units in an automated manner; and (v) reducing costs and times for high throughput when performing biological/medical assays. This may lead to improved clinical outcomes by allowing clinicians to detect protein expression levels in clinical subjects that: experience severe monthly menstrual and pelvic pain due to endometriosis, experience in vitro pregnancy loss, preeclampsia and preterm delivery; and undergo implantation failure during in-vitro fertilization (IVF)/ART. The medical testing devices herein detect and quantify cytokines, chemokines, acute phase reactants and growth factors that may mediate obstetric and gynecologic events such as early maternal-to-embryonic dialogue and endometrial immunological tolerance that eventually enables the semi-allogeneic blastocyst to implant in the uterus, menstrual cycle and menopause.

Interleukin 1 (IL-1) is a family of cytokine proteins which impacts the regulation of immune and inflammatory responses and sterile insults. The interleukin 1 (IL-1) family has 11 members of which interleukin 1-α (IL-1α) and interleukin 1-β (IL-1β) are the most studied in relation to parturition. In contrast to IL-1α, interleukin 1-β expression is inducible and not constitutive. Interleukin-1β is produced by hematopoietic cells, such as dendritic cells, blood monocytes, T cells, tissue macrophages, uterine tissues, and cells including myometrial smooth muscle, and placenta. The induction of IL-1β transcription is triggered by microbial stimuli, damage associated molecular patterns (DAMPs) from maternal tissues under progressively increasing physical stress as gestation advances, or lung surfactants released from the maturing fetus, via toll-like receptor 4 (TLR4), or pro-inflammatory cytokines. In addition, numerous exogenous agents (e.g., microbial components and asbestos) and endogenous agents (e.g., monosodium urate and calcium pyrophosphate dihydrate crystals) can activate caspase 1, which induces release of bioactive IL-1β.

The receptors for IL-1 comprise extracellular domains and the IL-1 receptor domain in the cytoplasmic portion of cells. Interleukin-1α, IL-1β, and IL-1 receptor antagonist (IL-1Ra) have two receptors: IL-1R1, which is biologically active, and IL-1R2, which lacks a signaling-competent cytoplasmic tail and acts as a decoy. There is also a soluble form of IL-1R2. IL-1R1 is activated once the IL-1 receptor accessory protein (RAcP) binds IL-1 and IL-1R1 to form a complex that activates intracellular adapter molecules. IL-1RAcP has an alternatively spliced isoform that varies in the C-terminal region termed IL-1RAcPb because it is primarily expressed in the central nervous system (CNS).

IL-1 receptor antagonist (IL-1Ra, which is also referred to as IL-1RN) is an acute phase reactant and regulates IL-1β activity. IL-1Ra binds to both IL-1R1 and IL-1R2 with high affinity and prevents signal transduction by IL-1α or IL-1β. A soluble form of the IL-1 receptor accessory protein (sIL-1RAcP) enhances IL-1α/β binding to IL-1R2 by approximately 100-fold, while leaving unaltered the low binding affinity of IL-1Ra and thereby preventing inflammation. Soluble AcP is present in normal human serum at an average concentration greater than 300 nanograms (ng)/milliliter (ml). Levels of circulating IL-1Ra and of cellular IL-1R2 therefore determine whether a pro-inflammatory response will begin, persist, or cease.

In humans, the locations for genes coding for both IL-1β and IL-1Ra reside near each other on the long arm of chromosome 2. The IL-1RA gene has a penta-allelic polymorphic site within the second intron as a result of having two to six copies of an 86 base pair (bp) tandem repeat. The variable number of tandem repeat (VNTR) region contains three potential protein-binding sites and is believed to have functional significance. A1 is the most common allele, which has four repeats. A2 is a less common allele, which contains two repeats and has been associated with increased production of IL-1Ra protein; reduced production of IL-1α protein by monocytes; and increased IL-1β activity. The clinical significance has been described in various diseases of autoimmune or inflammatory nature, such as vulvar vestibulitis, inflammatory bowel disease, multiple sclerosis, psoriasis, and lichen sclerosus. Allele 3 has five repeats, which has a frequency of about 1.0-3.5%. Allele 4 has three repeats, which has a frequency of about 0.7%.

IL-1α and IL-1β levels change during the course of the menstrual cycle. IL-1RII expression level changes throughout the course of menstrual cycle; with elevated levels during the late secretory phase of premenstrual and menstrual periods and decreased expression within the implantation period Immune system imbalances have been implicated in several obstetric and gynecologic diseases such as preterm delivery, pre-eclampsia, endometriosis, recurrent implantation failure (RIF) and recurrent pregnancy loss (RPL).

There is a growing interest in using immune-modulating treatments in women with endometriosis to correct potential immune imbalances. Current treatment modalities used in the context of endometriosis are hormones that negatively affect the woman's fertility or surgical removal of the lesions which is temporary and invasive.

### EXAMPLES

Aspects of the present teachings may be further understood in light of the following examples, which should not be construed as limiting the scope of the present teachings in any way. Data from women with endometriosis suggest that IL-1 system and IL-1Ra are involved in the pathogenesis and spread of endometriosis as well as the consequences (such as strictures and pain). The measurement of IL-1β and IL-1Ra levels by the medical testing device herein can be conclusive for diagnosing women with endometriosis across different genetic profiles.

### Example 1. Medical Devices Used in Making Measurements Pertinent to Chemical and Toxicological Parameters

The medical testing device herein may support tests comporting to the conventional testing paradigm and the functional testing paradigm. In the conventional testing paradigm, samples are obtained from a clinical subject/patient upon the onset of symptoms associated with an illness. In the functional testing paradigm, samples are obtained from a clinical subject/patient prior to the onset of symptoms associated with an illness. For example, the medical testing device may analyze samples to determine and monitor the following: (i) immune-responses to biological stimuli; (ii) levels of IL-1 proteins which may be associated with abnormal menstrual cycle; (iii) diagnosis for disease states associated with IL-1 and other diseases; (iv) diagnosis for conditions where IL-1 proteins are the underlying pathology for a medical condition; (v) diagnosis for diseases where IL-1 disorders are reported in the pathogenesis of clinical subjects/patients with endometriosis; (vi) cytokine responses where peripheral venous blood is collected in heparinized tubes with a specific stimulant; and (vii) induced levels of IL-1Ra may be measured in women with a history of menstrual and/or pelvic pain, and/or dyspareunia.

FDA Title 21 Chapter 1 SubChapter H-Medical Devices, Part 866—Immunology and Microbiology Devices, Subpart F-Immunological Tests, Sec. 866.5700 identifies whole human plasma or serum immunological test system as a device comprising reagents used to measure by immunochemical techniques the proteins in plasma or serum. Measurements of proteins in plasma or serum aid in the diagnosis of any disease concerned with abnormal levels of plasma or serum proteins (e.g., agammaglobulinemia, allergies, multiple myeloma, rheumatoid vasculitis, and hereditary angioneurotic edema.) These medical devices are classified as Class I general controls, and is exempt from the premarket notification procedures in subpart E of part 807 of this chapter subject to the limitations in 866.9. Supernatant/Serum induced IL-1Ra measurement is an immunologic test and may be used for screening at risk-patients/subjects and may be categorized as Class 1 and exempt. The medical devices used herein may be used in screening or diagnosing clinical subjects/patients with endometriosis where disorders of IL-1 systems are the underlying pathology.

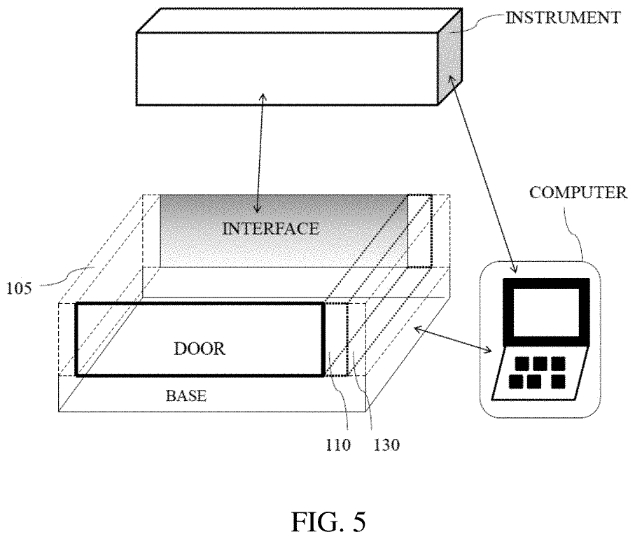

In FIG. 5, the medical testing device herein contains the base, door region, interface region, wall 105, wall 130, and control unit 110. The medical testing device herein is depicted as being connected to a computer and instruments as accessories for performing ELISA, ELISPOT, flow cytometry; polymerase chain reactions (PCR); photometry/spectrophotometry; protein interactions (e.g., DNase footprinting assay, filter binding assay, and get shift assay); a secretion assay for identifying cells secreting a protein (e.g., cytokine); and colorimetric protein assays (e.g., multiple Luminex bicinchoninic acid assay). The medical testing device herein includes a base that may contain units for providing magnetic fields, electric connections, heating actuators, rotating actuators, and shaking actuators. Wall 105, wall 130, and control unit 110 may be depicted as three-dimensional slices physically fastened/connected to the base. Control unit 110 is connected to wall 130, wherein control unit 110 contains electrical connection and physical ridges that control the movement of robotic transfer units. Wall 105 is separated from control 110 by the door and interface regions. The interface region of the medical testing device is a position near which the instrument resides. The instrument may be, but not limited to: equipment for flow cytometry; multiplex and other technologies assess induced levels of IL-1α, IL-1β, and IL-1Ra; automated machines for separating PBMC; incubators; and spectrophotometers. The computer is connected to the base of the medical testing device and instrument. Data obtained from the instruments may be sent to the computer for processing, such as calculating calibration curves. Additionally, the computer may be used to instruct control unit 110 to perform certain functions and implement certain protocols, such as transferring 30-microliters to a plate via a robotic transfer unit (described in more detail in below); transferring ELISA plates to the instrument; applying magnetic fields of certain strengths; shaking test tubes at specified revolutions per minute; and analyzing the measurement data.

Control unit 110 may attach to robotic transporters, which safely handles and moves test tubes, pipettes, syringes, and other materials used for diagnostic testing and measurements. In combination with separation and stimulation instruments, control unit 110 may lead to high throughput outcomes for measuring levels of protein and other biomolecules via ELISA, ultraviolet(UV)/visible(Vis) spectroscopy; fluorescence spectroscopy; absorbance values of label samples; flow cytometry, and reverse transcriptase polymerase chain reaction (RT-PCR). The separation unit may be just a specific test tube with a liquid gradient to separate the cells of interest. The stimulation unit may be a specific test tube with a stimulant in it specific for that disease and whole blood may be stimulated in the test tube. Control unit 110 may aid in or directly perform the following separation techniques: plastic/adhesion (based on surface charge and adhesion characteristics); density gradient centrifugation (based on cell density); filtration (based on cell size); selective cell culture (based on cell physiology and morphology); cell surface markers; aptamer technology; magnetic separation; microfluidics; and single-cell isolation (e.g., single-cell printers). The separations remove debris and other components of a sample extraneous to the target system of interest. Upon performing the separation techniques, a supernatant phase resides over the debris and extraneous components phase. The target system (e.g., IL-1 proteins implicated in pre-term deliveries) may be contained in the supernatant phase. A portion of the supernatant phase is sent to the instrument for analysis and measurement. RNA may be isolated using the cells for quantitative RT-PCR or flow cytometry may be conducted to measure the proteins inside the cells.

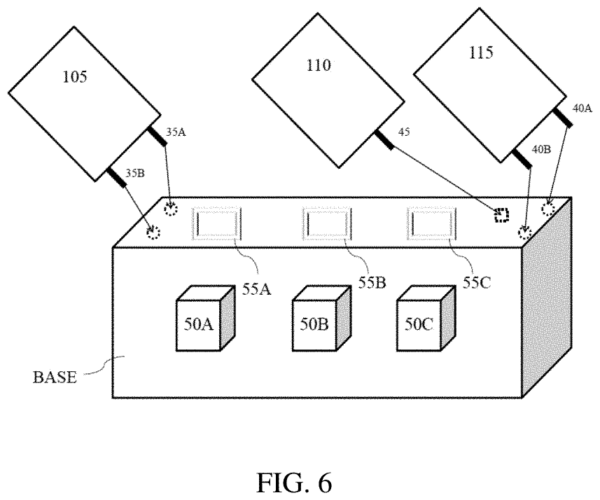

In FIG. 6, chambers 55A, 55B, and 55C contain movable ridges that are above the surface of the base. Test tube racks and n-well plates (n=1 to 384) may be accommodated by chambers 55A, 55B, and 55C. More specifically, these test tube racks and n-well plates may make a supple fit into chambers 55A, 55B, and 55C. Units 50A, 50B, and 50C may be a combination of electric, heating, magnetic, and rotating units in the base. Electric currents, heat, magnetic fields, and spinning from units 50A, 50B, and 50C may be applied to test tube racks and n-well plates accommodated in chambers 55A, 55B, and 55C. Connectors 35A and 35B of wall 105 may fit into the base at the indicated dotted circles, as denoted by arrows. Connectors 40A and 40B of wall 130 may fit into the base at the indicated dotted circles, as denoted by arrows. Connectors 35A, 35B, 40A, and 40B may latch to the base to create stable connections to keep wall 105 and wall 130 in an upright position. Connector 45 of control unit 110 may be a physical connector and electrical plug fitting into the base at the indicated dotted box, as denoted by the arrow. The electric unit among units 50A, 50B, and 50C may power control unit 110 via connector 45.

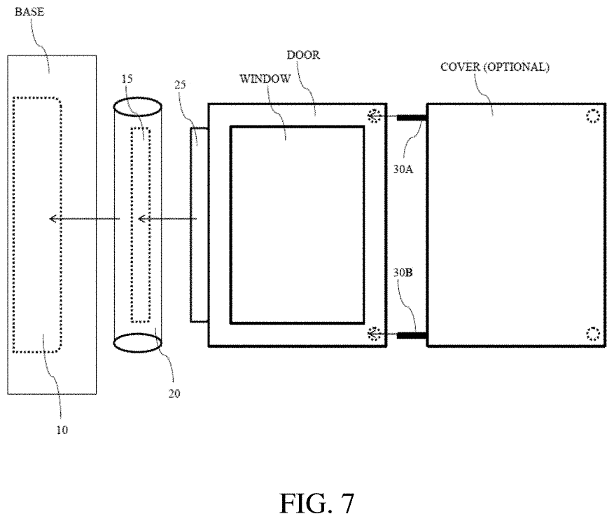

In FIG. 7, the door and top cover units are depicted. The sample processing needs to be done under sterile conditions. The incubation for stimulation needs to be done at 37° C. 5% CO2. The incubation for stimulation may be done on heating blocks that shake. The base may have inner chamber 10. Rolling connector 20 may have inner chamber 15. The door may have a window and has connector 25. The optional cover, which may have connectors 30A and 30B, fits over the top of the medical testing device. Connectors 30A and 30B of wall 130 may fit into the door at the indicated dotted circles, as denoted by arrows. The door and option cover may be perpendicular to each other upon connection. Connector 25 may fit into inner chamber 15 at the indicated dotted rectangle, as denoted by the arrow. Rolling connector 20 may be a cylinder which allows the door to move up and down when attached to base. Rolling connector 20 may fit into inner chamber 10 in the base at the indicated rectangle, as denoted by the arrow.

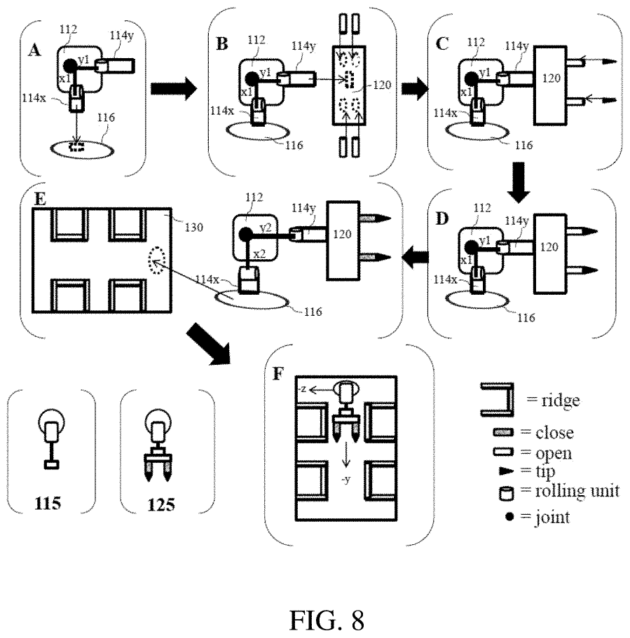

In FIG. 8, insets A-F depict the assembly of the first robotic transfer unit. Slider 116 may be a wheel. Hand 112 may contain a joint (depicted as the bold dot), wherein the joint contains an x-arm and y-arm. The x-arm and y-arm can vary in length, and thus be contracted or extended in length. The x-arm may connect to connector 114x via a rolling unit. The y-arm may connect to connector 114y via a rolling unit. The rolling unit may allow components attached to connectors 114x and 114y to rotate 360 degrees. The length of x1 of the x-arm is less than the length of x2. The length of y1 of the y-arm is less than the length of y2.

In inset A of FIG. 8, connector 114x (where x is associated with the x-direction) may attach to slider 116 at the dotted box, as indicated by the arrow. The resulting assembly from inset A of FIG. 8 may be assembly 115. In inset B of FIG. 8, connector 114y (where y is associated with the y-direction) may attach to holder 120. Holder 120 may support volume transferring equipment, such as pipetting bodies and syringe bodies. The transferring equipment may be in an open state (as depicted as an un-shaded cylinder) or close state (as depicted as shaped cylinder). The volume transferring equipment may attach to holder 120 at the dotted circles, as indicated by the arrows. In inset C of FIG. 8, disposable and removable tips, such as tips for pipettes and needles for syringes, may attach to the transferring equipment in the open state. In inset D of FIG. 8, the x-arm and y-arms may be extended from x1 to x2 and y1 to y2. The resulting assembly from inset D going to inset E in FIG. 8 may yield robotic transporter 125, where the transferring equipment is in the close state. Assembly 125 in inset E in FIG. 8 may connect to wall 130 at the dotted circle via slider 116. Wall 130 may have ridges on the surface of the wall which may be exposed or covered. The ridges may create a path for guiding robotic transporter 125. In inset F in FIG. 8, robotic transfer unit 125 may move along the ridges in the x-direction or y-direction. Robotic transporter 125 has an x-arm which allows for movement in the x-direction. As indicated above, the rolling unit and circular shape of slider 116 may allow for 360 degree motion and movements.

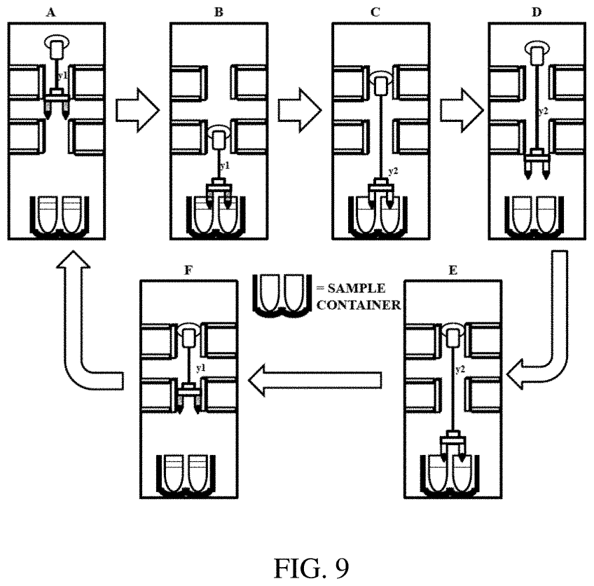

In FIG. 9, insets A-F depict the motions of robotic transporter 125, where y2 is a greater length than y1. In inset A of FIG. 9, robotic transporter 125 may be above the top and the bottom ridges where the transferring equipment in the close state and the length of the y-arm is of y1. In inset B of FIG. 9, robotic transporter 125 may be lowered to a position in between the bottom ridges. The tip is in the liquid within the sample container. In inset C of FIG. 9, the tip may remain within the sample container such that liquid is taken up into the transferring equipment. The length of the y-arm may be extended to y2, where the top portion of robotic transporter 125 (i.e., slider 116, joint, and connector 114x) is elevated to a position in between the top ridges. The liquid layer in inset C of Fig. C is now the dotted line level. In inset D of FIG. 9, robotic transporter 125 is elevated to a position above the top ridges, where the length of the y-arm is y2 and the transferring equipment is in the open state containing fluid from the sample container. In inset E of FIG. 9, robotic transporter 125 may be lowered to a position between the top two ridges, where the length of the y-arm is y2 and the transferring equipment is in the open state containing fluid from the sample container. Thereby, the tip may be in the sample container to ensure liquid released from the tip transfers back to the sample container. In inset F of FIG. 9, the top portion of robotic transporter 125 may remain in the same position as the top portion of robotic transporter in inset E of FIG. 9. The length of the y-arm may be contracted from y2 to y1 and thereby releasing fluid back to the sample container, as depicted the solid liquid line in the sample container. Additionally, the transferring equipment is transformed back to the close state. While not explicitly depicted, robotic transporter 125 can extend along the x-arm to transfer or uptake liquid in other sample containers.

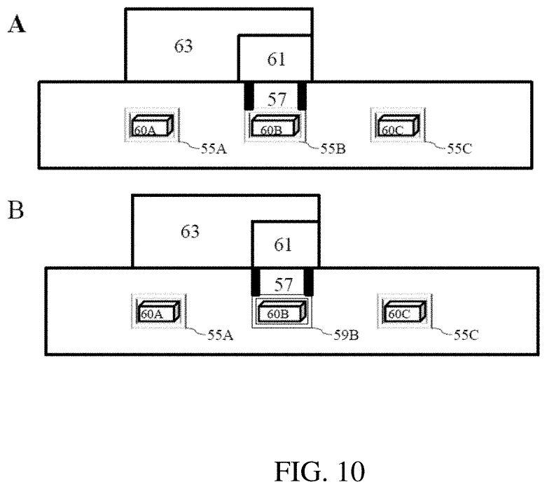

In FIG. 10, insets A and B depict the first part of a pushing mechanism supported by a robotic transporter unit. Unit 61 and unit 63 may be the receiving region and operational region of an instrument. If the instrument is a cytometry machine, then unit 61 is where samples are placed and unit 63 is the operational region comprising a flow cell, a measuring system, a detector, and an amplification system. A computer (as depicted in FIG. 5) for analysis of the signals has been omitted from FIG. 10 for purposes of clarity. More specifically, the flow cell for unit 63 has a liquid stream (sheath fluid) for carrying and aligning the cells so that they pass single file through the light beam for sensing. The measuring system commonly uses measurement of impedance (or conductivity) and optical systems—lamps (mercury, xenon); high-power water-cooled lasers (argon, krypton, dye laser); low-power air-cooled lasers (argon (488 nm), red-HeNe (633 nm), green-HeNe, HeCd (UV)); diode lasers (blue, green, red, violet) resulting in light signals. The detector and analog-to-digital conversion (ADC) system converts analog measurements of forward-scattered light (FSC), side-scattered light (SSC), and dye-specific fluorescence signals into digital signals that can be processed by a computer. The amplification system can be linear or logarithmic.

In inset A of FIG. 10, samples are contained in container unit 60C, which resides in chamber 55C. Assay unit 60B may be an n-well plate for ELISA or other assays, which resides in chamber 55B. Waste unit 60A may be test tube racks for swapping used pipettes or syringe tips with new pipette or syringe tips, which reside in chamber 55A. Path 57 may be outlined by ridges on the surface of the base to create a path between chamber 55B and unit 61. In other embodiments, there may be multiple versions of path 57, where path 57 may be between other chambers and portions of the instrument. In inset B of FIG. 10, chamber 59B is topologically different from chamber 55B in that the ridges of chamber 55B are flattened to surface level of the base. The outer perimeter of the ridges is thereby at the same surface as the base. The width of path 57 can accommodate assay unit 60B.

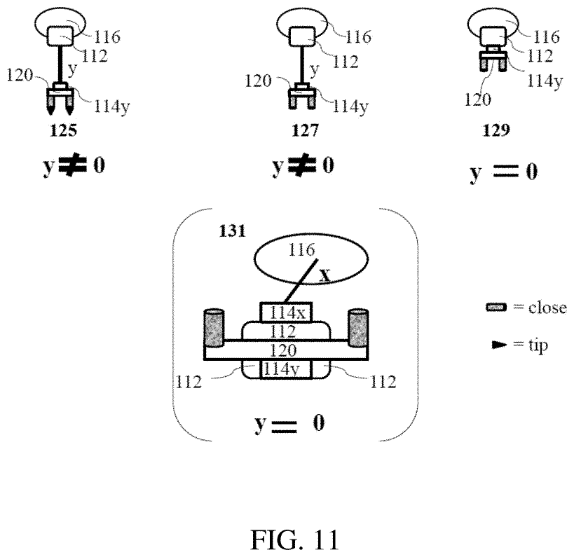

In FIG. 11, robotic transporter 125 is oriented such that liquid volumes may be transferred to test tubes or wells. Removal of tips from robotic transporter 125 may yield assembly 127, where the y-arm has a length greater than 0 distance units. The y-arm may be contracted to a length of 0 distance units, such that there is no separation between connector 114y and hand 112 in assembly 129. Holder 120, the transferring equipment attached to holder 120, and connector 114y may be folded up 180 degrees to yield robotic transporter 131.

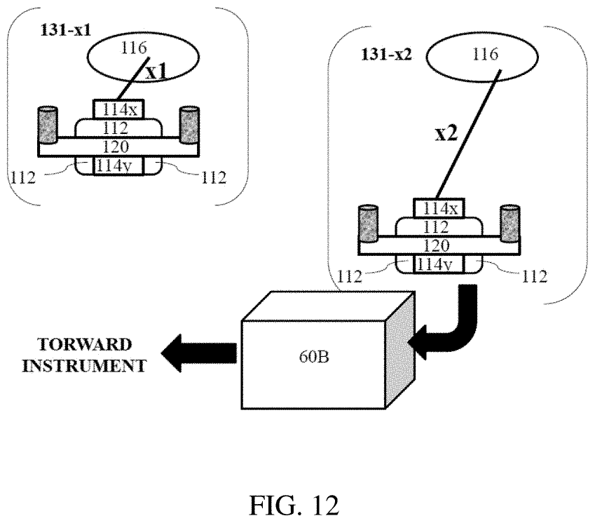

In FIG. 12, the pushing mechanism implemented by robotic transporter 131 is depicted. If the x-arm is x1, then the length of the x-arm is not long enough to reach assay unit 60B. If the x-arm is x2, then the length of the x-arm is long enough to reach assay unit 60B. Along connector 114x, the rolling joint may allow connector 114x, connector 114y, hand 112, and holder 120 to be rotated. The resulting rotations may allow these components to face and touch the side of assay unit 60B, as pointed to by the curved bold arrow. As slider 116 moves along the (−)-z-axis, robotic transporter 131 may push/move assay unit 60B toward the instrument, which is also in the (−)-z-direction.

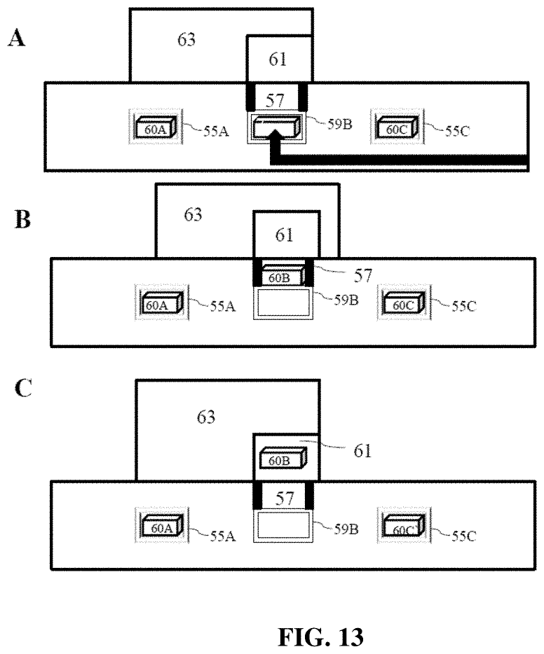

In FIG. 13, insets A-C depict the pushing mechanism implemented by robotic transporter 131. In inset A in FIG. 13, robotic transporter 131 may move in the x-direction and the −z-direction towards chamber 59B. In inset B in FIG. 13, assay unit 60B may be subsequently moved to path 57. In inset C in FIG. 13, assay unit 60B may be subsequently moved to container unit 61.

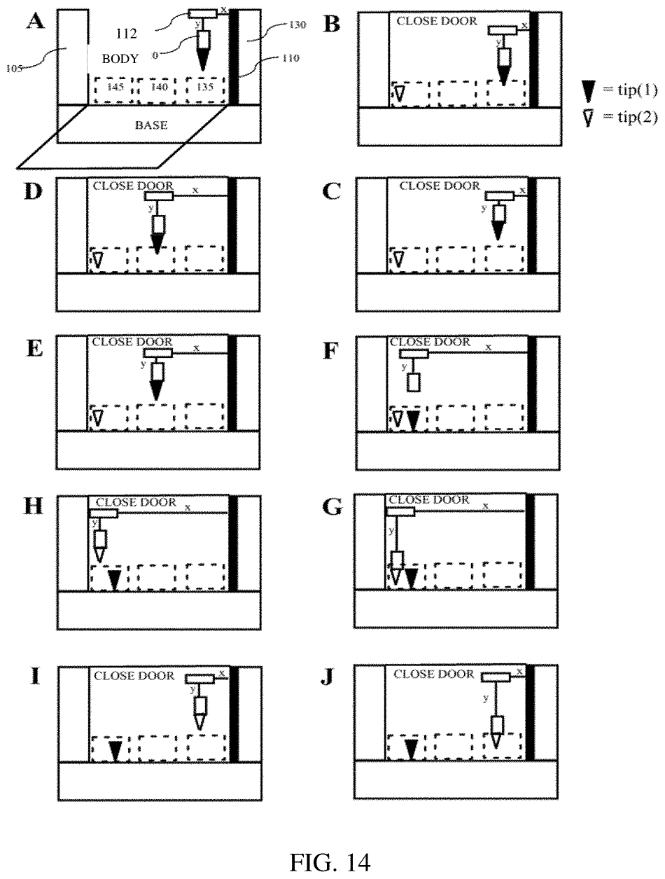

In FIG. 14, insets A-J depict the automated tip changing mechanism by robotic transporter 125. The computer (from FIG. 5) may be programmed to instruct that the robotic transporter 125 is not activated until the door is closed. Robotic transporter 125 is depicted as the x-arm, y-arm, hand 112, the body of transferring equipment (syringe or pipette), and accompanying tip. The dark-shaded inverted triangle represents “tip(1)” and the un-shaded inverted triangle represents “tip(2)”. Region 135, which is proximal to control unit 110, may contain samples of interest that are being tested for IL-1 protein (which is akin to container unit 60C in FIG. 13). Region 145, which is proximal to wall 105, may contain racks for new tips, such as tip (2) (which is akin to waste unit 60A in FIG. 13). Region 140, which is in between regions 135 and 145, may contain the assay unit for ELISA or PCR (which is akin to assay unit 60B in FIG. 13).

In inset A of FIG. 14, the door is open in the medical testing device and thus robotic transporter 125 is not activated. In insets B-J of FIG. 14, the door is closed in the medical testing device and thus robotic transporter 125 is activated. In inset B of FIG. 14, tip(1) may be lowered into region 135 to collect liquid volumes from samples. In inset C of FIG. 14, tip(1) may collect the liquid volume as the y-arm contracts. In inset D of FIG. 14, the x-arm may extend over to region 140 and the y-arm may extend into region 140. The liquid volume from tip(1) is transferred to region 140. In inset E of FIG. 14, the x-arm may remain extended over region 140 and the y-arm may contract. In inset F of FIG. 14, the x-arm may extend over to the right hand portion of region 145 and the y-arm may extend to release tip(1). In inset G of FIG. 14, the x-arm may extend further over to the left hand portion of region 145 and the y-arm may extend to latch onto tip(2). In inset H, the x-arm may remain extended over region 145, the y-arm contracts, and tip(2) is attached to robotic transporter 125. In inset I, the x-arm may contract back over region 135, where tip(2) is now above region 135. In inset J, tip(2) may extend into region 135 as the y-arm extends.

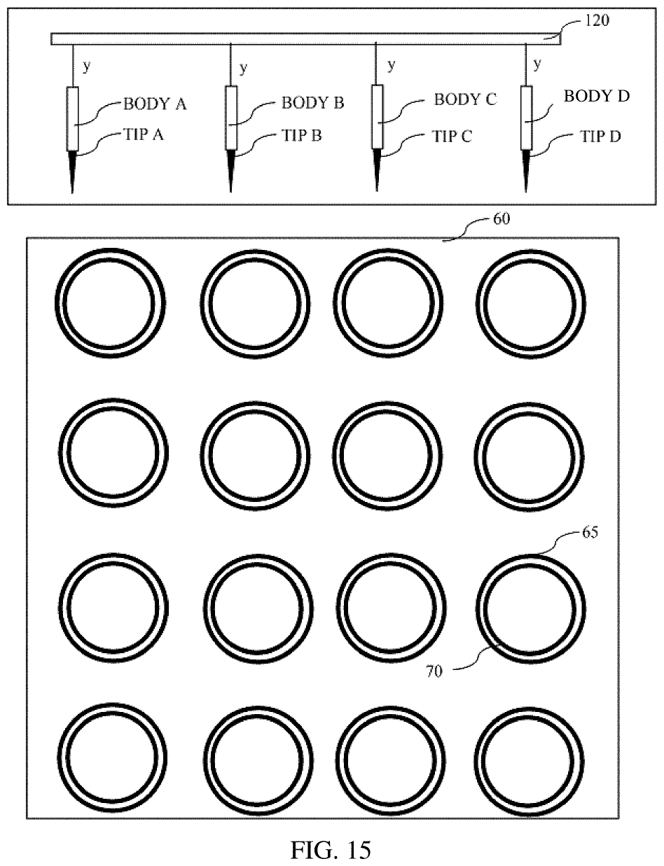

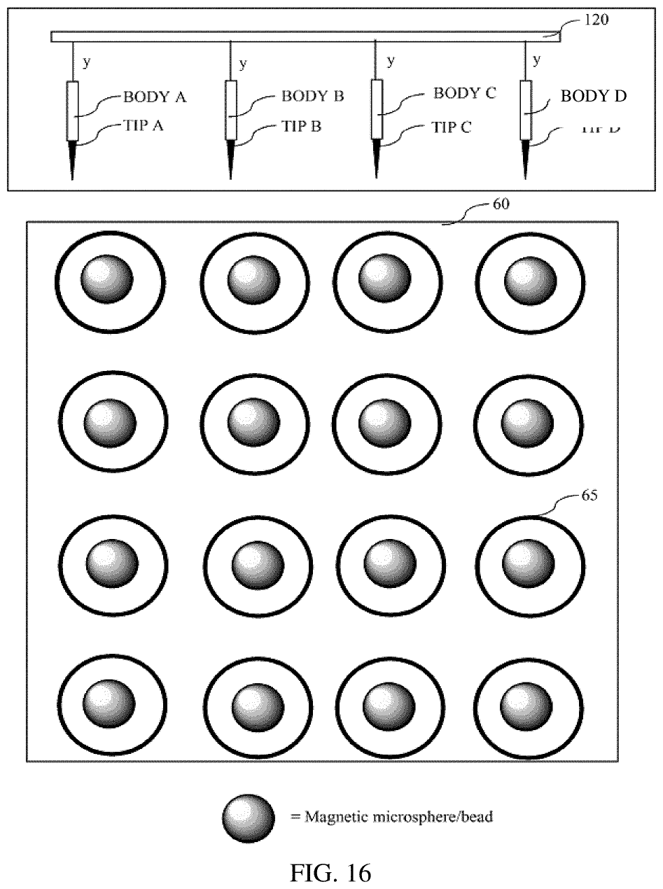

In FIG. 15 and FIG. 16, the plates (plate 60) are a 4×4 plate with sixteen wells equipped for immunomagnetic separation (IMS). The IMS may isolate cells, proteins, and nucleic acids within a cell culture or body fluid via specific capture of biomolecules. In FIG. 15, plate 60 is a 4×4 plate with sixteen wells. The inner perimeter of magnet 70 may make a supple fit with a test tube or a PCR tube. The outer perimeter of magnet 70 may make a supple fit with perimeter 65 of each well.

In an example, blood is drawn directly into tubes with stimulant(s) or control(s). The tubes may be enclosed in cases at 37 degrees Celsius and transferred to the medical testing device to analyze IL-1 system component levels (IL-1α, IL-1β, and IL-Ra). IL-1 function may be calculated as the fold increase over that seen in control treated tubes. These tubes may be placed in each well that is equipped with magnet 70. Magnet 70 is slightly shorter in length than the length of these tubes. The pipette bodies (body A-D) and tips (tips A-D) attached to holder 120 transfer stimulants or biological entities to the tubes placed in each well, wherein each well is equipped with magnet 70. The magnetic field generated by the base of the medical testing device herein may lead to efficient separation of the supernatant phase. The supernatant phase can be removed by the pipette bodies (body A-D) and tips (tips A-D) attached to holder 120.

Small-magnetized particles and beads, such as beads in FIG. 16, may contain antibodies and lectins. In FIG. 16, the beads are super paramagnetic materials that may be coated with primary antibodies to bind to targeted IL-1 proteins. The targeted IL-1 proteins may be gently separated and go through multiple cycles of washing cycles performed by the pipette bodies (Body A-D) and tips (tips A-B) attached to holder 120. This may obtain the target IL-1 protein bound to these super paramagnetic beads. The bound IL-1 proteins can be differentiated, based on strength of magnetic field. IL-1 proteins can be eluted to collect supernatant and the concentration of specifically targeted biomolecules can be determined. Additionally, the IMS may obtain certain concentrations of specific molecules within targeted bacteria. The magnetic beads may be coated with primary antibodies, specific-specific antibodies, lectins, enzymes, or streptavidin. The magnetic beads comprise iron-containing cores covered by a thin layer of a polymer shell allowing for the absorption of proteins and other biomolecules. The linkages between magnetized bead-coated materials may be cleavable DNA linker cell separation from the beads when the culturing of cells is more desirable. Many of these magnetized beads have the same principles of separation. However, the presence and different strengths of magnetic fields require certain sizes of beads, based on the ramifications of the separation of the cell population.

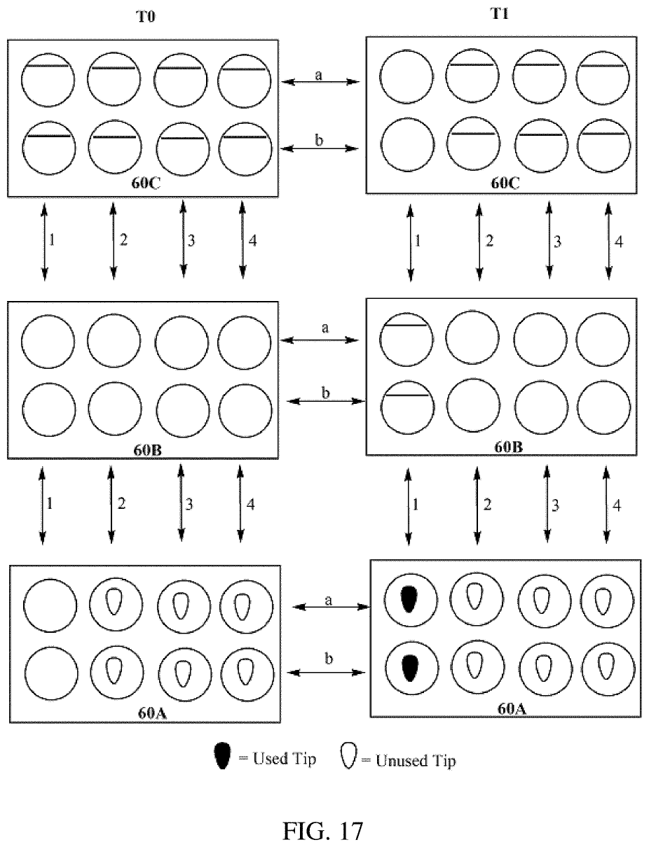

FIGS. 17-20 are depictions of the transfer of volume of samples and the disposal of tips at different times.

In FIGS. 17-20, units 60A, 60B, and 60C are each plates with 2×4=8 wells. The rows are “a” and “b” and the columns are “1”, “2”, “3”, and “4”. Unit 60C is where the sample is collected and transferred to unit 60B for ELISA, PCR, or other assay. Unit 60A is where used tips are replaced with clean tips. In other examples, needles or volume transferring components may be used.

At T0 in FIG. 17: All of the wells have sample to be transferred from unit 60C. None of the wells in unit 60B have samples transferred. Wells a1 and b1 are empty in unit 60A, whereas wells a2-a4 and b2-b4 have unused pipette tips.

At T1 in FIG. 17: Sample has been transferred from a1 and b1 in unit 60C to a1 and b1 in unit 60B. Used pipette tips are dropped off at a1 and b1 in unit 60A.

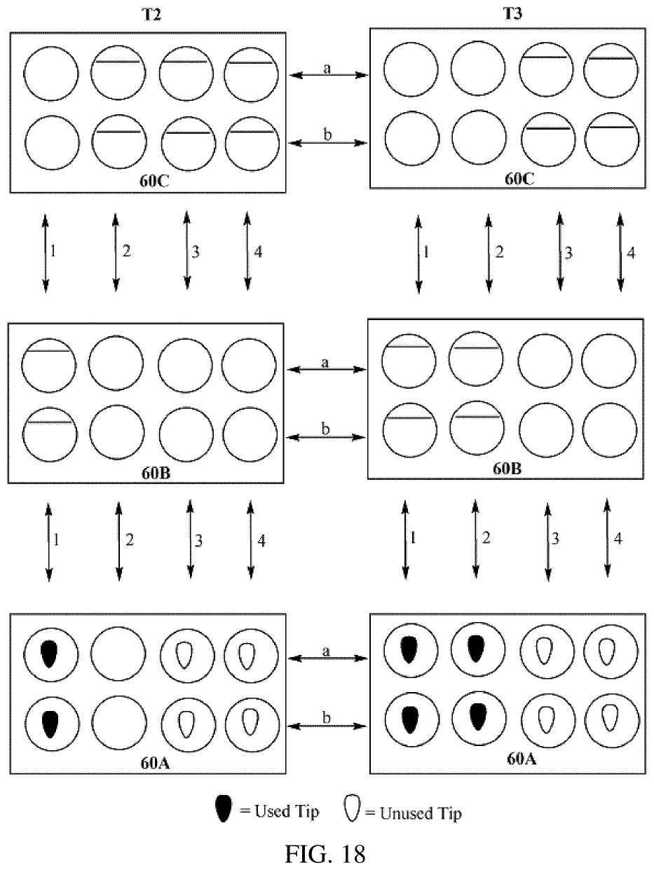

At T2 in FIG. 18: Unused tips from a2 and b2 from unit 60A are attached to the body of pipette and thus appear absent from a2 and b2 in unit 60A.

At T3 in FIG. 18: Sample has been transferred from a2 and b2 in unit 60C to a2 and b2 in unit 60B. Used pipette tips are dropped off at a2 and b2 in unit 60A. Prior used pipette tips remain in a1 and b1 in unit 60A.

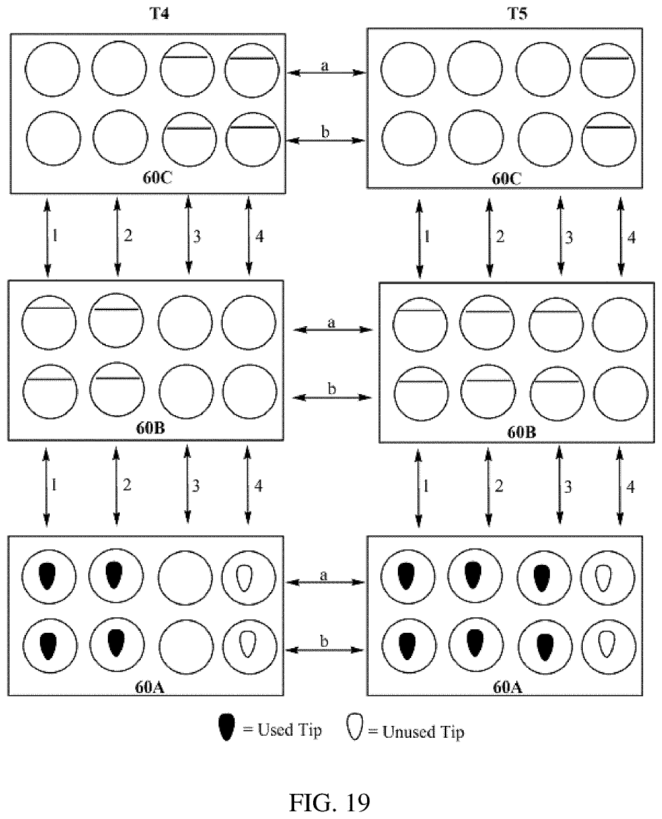

At T4 in FIG. 19: Unused tips from a3 and b3 from unit 60C are attached to the body of pipette and thus appear absent from a3 and b3 in unit 60C.

At T5 in FIG. 19: Sample has been transferred from a3 and b3 in unit 60C to a3 and b3 in unit 60B. Used pipette tips are dropped off at a3 and b3 in unit 60A. Prior used pipette tips remain in a1, a2, b1, and b2 in unit 60A.

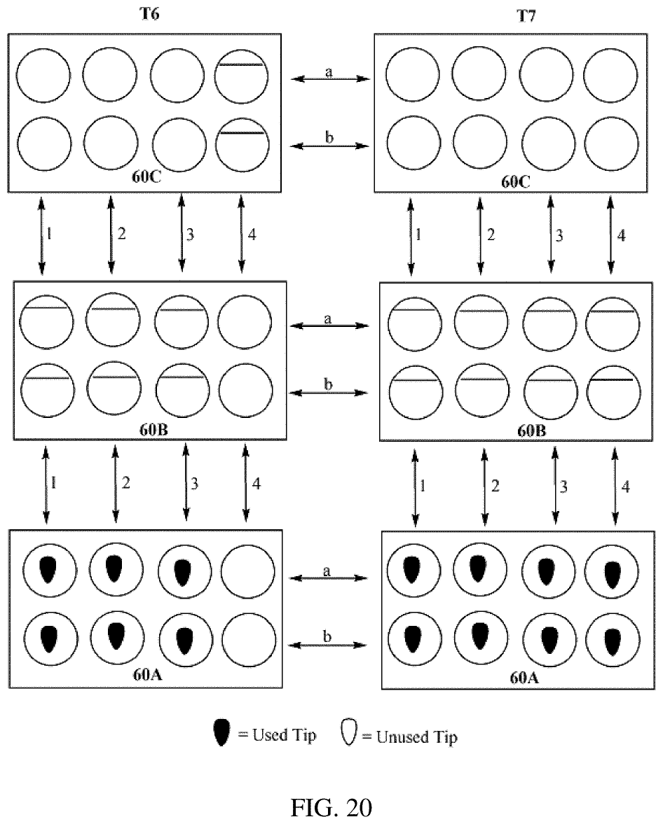

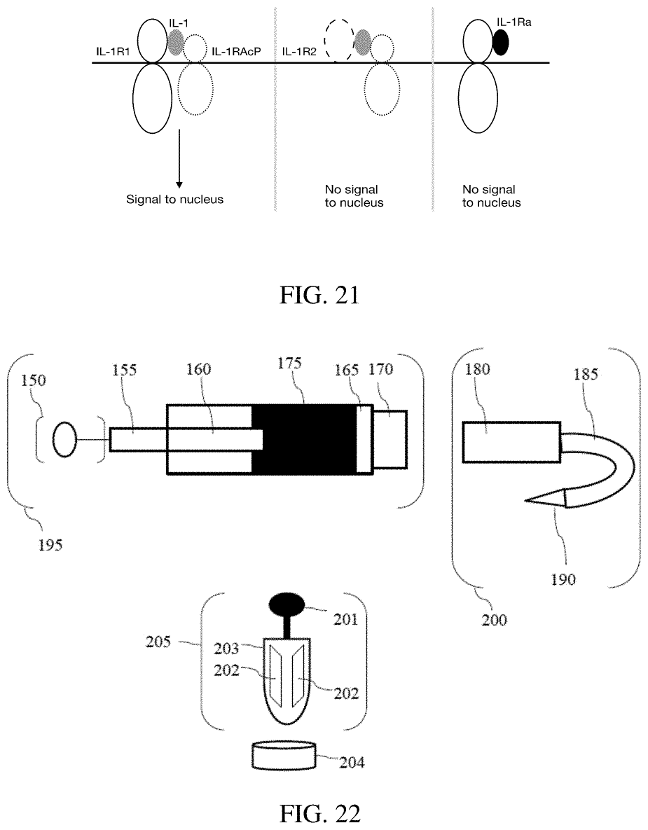

At T6 in FIG. 20: Unused tips from a4 and b4 from unit 60C are attached to the body of pipette and thus appear absent from a4 and b4 in unit 60C.

At T7 in FIG. 20: Sample has been transferred from a4 and b4 in unit 60C to a4 and b4 in unit 60B. Used pipette tips are dropped off at a4 and b4 in unit 60A. Prior used pipette tips remain in a1, a2, a3, b1, b2, and b3 in unit 60A.

### Example 2. Induced IL-1 System Measurement May be Used for Diagnostic Screening Purposes

Stimulated IL-1Ra may be used as a biomarker. The IL-1 system has been shown to play a role in obstetrics (pregnancy) and gynecologic (endometriosis) diseases. However, straight measurement of IL-1 system components in blood or genetic testing has not been conclusive and cannot be used for diagnostic purposes. By using the medical testing device herein, a new method can conclusively assess the IL-1 system for screening or diagnostic purposes directed to preterm delivery and endometriosis.

Scenario 1: Although IL-1Ra and IL-1 abnormalities have been shown to play a role in preterm delivery, for women who deliver preterm and are otherwise healthy, drawing blood, and routine immune marker testing does not reveal any pathology, which can only be uncovered when the immune system is induced/stressed.

Clinical Studies Directed to Scenario 1: For clinical studies, blood is drawn from women with history of preterm (n=3 clinical subjects/patients) or full term delivery (n=3 clinical subject/patients). In a laboratory, LPS stimulates peripheral blood mononuclear cells (PBMC) and immune responses are assessed using Biorad-Bioplex proteomic assay. The Biorad-Bioplex proteomic assay is performed using the medical testing device disclosed herein. The clinical studies enrolled women of all ethnicities and history of (h/o) singleton delivery. In contrast, the clinical studies does not enroll: women with a history of multiple pregnancies; women delivering less than 6 months prior to the enrollment; women lactating; women with autoimmune disease, diabetes mellitus, cardiovascular disease, severe obesity, major depression, and obstetric risk conditions (e.g., systemic maternal disease, placental or cord abnormalities, uterine anomalies, congenital malformations, chromosomal abnormalities, HIV, chorioamnionitis, acute or chronic illness of HPAG axis (hypopituitarism, adrenal insufficiency, polycystic ovary syndrome, etc.)); women recently engaging in chemical abuse (e.g., cocaine); and women engaging in drug use known to modulate HPAG-placenta and/or inflammatory axis (e.g., nicotine, corticosteroids, immune-suppressants, etc.).

Even though baseline PBMC produced IL-1Ra levels are similar between two cohorts, post-cortisol LPS stimulated PBMC from women who delivered full term produce more IL-1Ra compared to those from women with h/o preterm delivery (p=0.01). Post-cortisol LPS induced IL-10 and IL-13 levels are also higher among women who delivered full term when compared to those who delivered preterm. Pretreatment with cortisol did not accentuate these differences. In turn, the protocol is revised where PBMC is stimulated with LPS overnight. The supernatant cytokines (IL-10, IL-1Ra, and IL-13) are measured by enzyme-linked immunosorbent assay (ELISA) to identify the women at risk for preterm delivery. ELISA detects the presence of ligand (e.g., a protein) in a sample using antibodies directed against the protein to be measured.

Because LPS stimulation of PBMC did not induce IL-13, pilot experiments are conducted using phytohemagglutinin (PHA) as a stimulant. A cohort of 44 non-pregnant patients is enrolled (28 patients with history of full term and 16 patients with history of preterm birth). Similar inclusion and exclusion criteria are followed to enroll subjects. The resulting clinical data may suggest significant differences of ratio of Stimulated IL-10 to Stimulated IL-13 between Full Term and Preterm. Modeling indicated that a total of 275 patients are needed with approximately 32% (88 pts) with history of preterm.

| Pregnancy status | Full term | Preterm | Total |
|---|---|---|---|
| Nonpregnant | 139 | 26 | 165 |
| Pregnant | 42 | 9 | 51 |

The clinical studies enrolled pregnant women (n=51 clinical subjects/patients) and nonpregnant women (n=165 clinical subjects/patients) with a history of preterm or full term delivery and did not have any exclusion criteria. The median age is 34.00 years, where the minimum age is 19.00 years and maximum age is 45.00 years. The ethnicity of the women enrolled in the study is: 22% of African descent, 41% of Caucasian descent, 29% of Hispanic descent, and 8% of other. Note: age data is missing from 41 women and ethnicity data is missing from 28 women.

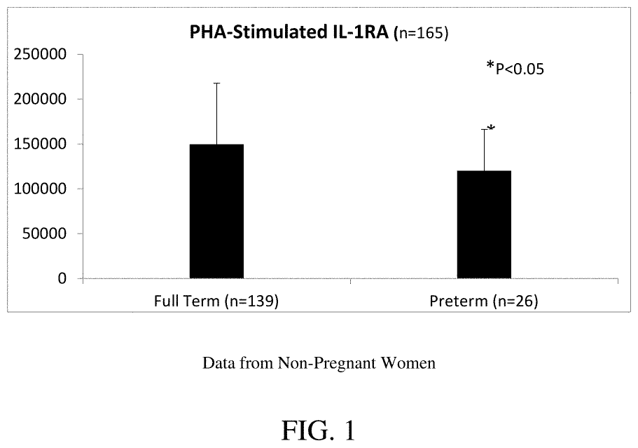

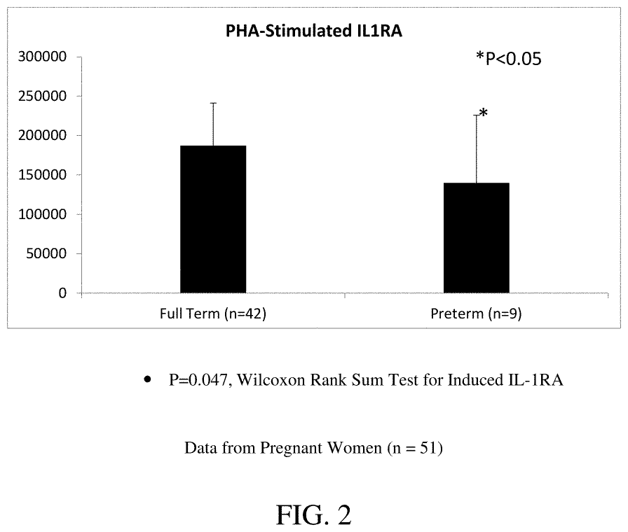

Blood collected by the obstetricians taking care of the subjects is sent to Kellbenx laboratory. PBMC is separated using Ficoll-Hypaque process, counted, and incubated initially with LPS. Due to low IL-13 production upon LPS stimulation (see FIG. 1), phytohemagglutinin (PHA) is subsequently used as the stimulant (see FIG. 2) at a concentration of 20 μg/ml. The stimulated PBMC obtained via PHA for healthy nonpregnant women with a history of preterm delivery produces lower levels of IL-1Ra in comparison to the stimulated PBMC obtained via PHA from women with a history of full term delivery. PBMC obtained from pregnant women with a history of preterm delivery (average 17.2 weeks ±8 weeks pregnancy at the time of blood draw) produce lower or higher IL-1Ra than that observed in pregnant women who delivered full term in the past. None of the pregnant women delivered preterm. Other gradients, filters, and automated systems may be used in combination with or in place of the Ficoll-Hypaque process. Medical devices, as described above, may perform a functional IL-1RA test for diseases where disorders of IL1 system are the underlying pathology.

Logistic regression analyses suggest that the preterm delivery history was not influenced by the subject's age or ethnicity.

| Predictor | N | OR | 95% CI | P-value | Comment |
|---|---|---|---|---|---|
| Pregnant | 213 | 1.38 | 0.61-3.11 | 0.47 | Can combine |
| | | | | | Pregnant & Non |
| Maternal Age | 172 | 1.08 | 0.72-1.62 | 0.71 | For a 5-year |
| | | | | | increase |
| Age ≥40 | 172 | 1.02 | 0.31-3.31 | 0.98 | |
| Ethnicity | 185 | | | 0.69 | Maybe too many |
| | | | | | categories |
| Black | | 1.46 | 0.47-4.53 | 0.52 | Versus Caucasian |
| Hispanic | | 0.68 | 0.19-2.38 | 0.55 | Versus Caucasian |
| Other | | 0.65 | 0.08-5.68 | 0.70 | Versus Caucasian |
| Caucasian v | 185 | 1.05 | 0.40-2.74 | 0.92 | |
| Non-Caucasian | | | | | |
| African v Non- | 185 | 1.73 | 0.61-4.87 | 0.30 | |
| African | | | | | |

From a clinical perspective, the ratio of induced IL-1Ra and induced IL-1Ra to baseline IL-1Ra from women with a history of preterm delivery is significantly different in comparison to that obtained from women with a history of full term delivery. From a clinical perspective, there is no significant difference in induced IL-10 or IL-13 between two cohorts of women. Ratio of Stimulated IL-10/Stimulated IL-13 is no longer statistically significant.

| Predictor | N | OR | 95% CI | P-value | Comment | Promise |
|---|---|---|---|---|---|---|
| Induced IL-10 | 213 | 1.01 | 0.98-1.03 | 0.64 | For a 100-unit | No |
| | | | | | increase | |
| Induced IL-1Ra | 213 | 0.991 | 0.983-0.998 | 0.0096 | For a 1000-unit | Yes |
| | | | | | increase | |
| Induced IL-13 | 213 | 1.01 | 0.98-1.04 | 0.64 | For a 100-unit | No |
| | | | | | increase | |
| Ratio IL-10 | 213 | 1.004 | 0.98-1.03 | 0.79 | For a 100-unit | No |
| | | | | | increase | |
| Ratio IL-1Ra | 213 | 0.88 | 0.78-0.995 | 0.041 | For a 10-unit | Yes |
| | | | | | increase | |
| Ratio IL-1Ra/IL-10 | 213 | 0.37 | 0.17-0.79 | 0.011 | For a 0.10-unit | Yes |
| | | | | | increase | |
| StimIL-13/RatioIL-10 | 213 | 1.28 | 0.81-2.00 | 0.29 | For a 100-unit | No |
| | | | | | increase | |

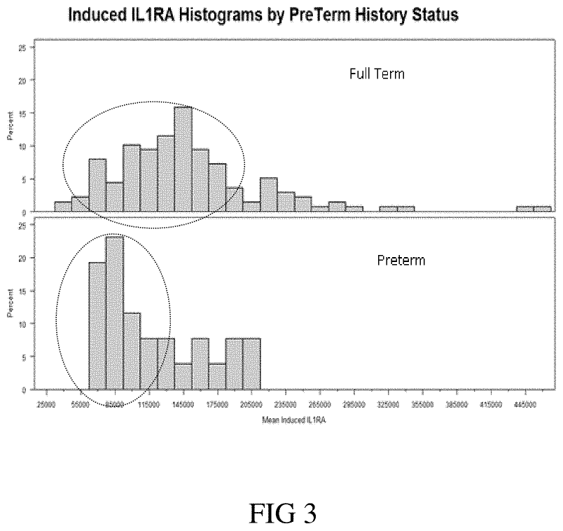

Based on the histogram in FIG. 3, the summarizing table below results. The normal range of induced IL-1Ra levels are indicated by the sections of the histogram encompassed by the dotted circles for preterm and full term clinical subjects. TP refers to true positive; FN refers to false negative; TN refers to true negative; FP refers to false positive; Sens % refers to sensitivity; and spec % refers to specificity.

| Cutoff≤ | TP | FN | TN | FP | Sens % | Spec % | PPV % | NPV % |
|---|---|---|---|---|---|---|---|---|
| 100000 | 14 | 12 | 110 | 29 | 53.8 | 79.1 | 32.6 | 90.2 |
| 125000 | 17 | 9 | 88 | 51 | 65.4 | 63.3 | 25.0 | 90.7 |
| 150000 | 19 | 7 | 56 | 83 | 73.1 | 40.3 | 18.6 | 88.9 |
| 175000 | 22 | 4 | 36 | 103 | 84.6 | 25.9 | 17.6 | 90.0 |
| 200000 | 24 | 2 | 23 | 116 | 92.3 | 16.6 | 77.1 | 92.0 |

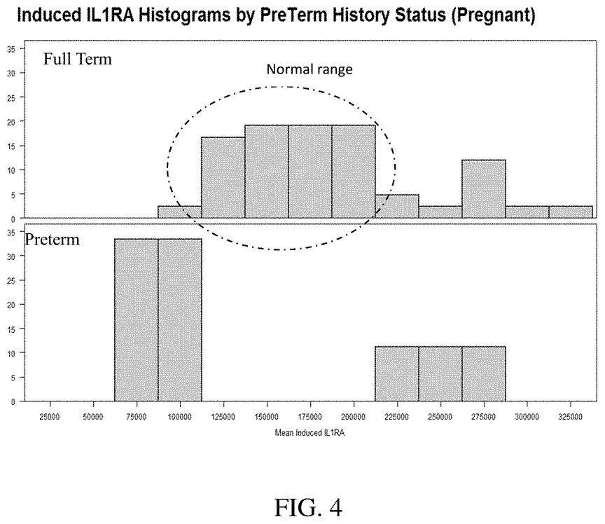

Based on the histogram in FIG. 4, the summarizing table below results. The normal range of induced IL-1Ra levels are indicated by the sections of the histogram encompassed by the dotted circle for full term clinical subjects.

| Cutoff≤ | TP | FN | TN | FP | Sens % | Spec % | PPV % | NPV % | Comment |
|---|---|---|---|---|---|---|---|---|---|
| 75000 | 3 | 6 | 42 | 0 | 33.3 | 100 | 100 | 87.5 | Only 3+ |
| 100000 | 5 | 4 | 42 | 0 | 5.6 | 100 | 100 | 91.3 | Only 5+ |
| 125000 | 6 | 3 | 38 | 4 | 6.7 | 90.5 | 60.0 | 92.7 | Only 10+ |
| 150000 | 6 | 3 | 29 | 13 | 6.7 | 9.1 | 31.6 | 90.6 | |
| 175000 | 6 | 3 | 23 | 19 | 6.7 | 4.8 | 24.0 | 88.5 | |
| 200000 | 6 | 3 | 15 | 27 | 6.7 | 5.7 | 18.2 | 83.3 | |

Other stimulants can be used as indicated in PCT Patent Publication WO 2011/071893.

Scenario 2: ELISA Testing: The medical testing device herein may facilitate high throughput ELISA testing in an automated and expedited manner A program on the computer may be configured to instruct the robotic transfer units (robotic transfer units 125 and 131) to perform certain functions, as described above.

In Scenario 2, ELISA detects the presence of ligand (e.g., protein, antibodies, and hormones) in a sample using antibodies directed against the protein to be measured. In Scenario 2, the ligand of interest may be IL-1 proteins contained in biological samples. The biological samples derive from clinical subject/patients, which may be a human or animal organism. The medical testing device herein may be used by a medical testing lab to implement standardized ELISA systems to measure IL-1 system elements including IL-1Ra. In combination with the medical testing device herein, six test tube racks (e.g., first, second, third, fourth, fifth, and sixth test tube racks), pipette tips, a 96-plate well, incubator, and spectrophotometer are used for this ELISA protocol. The incubator and spectrophotometer are examples of instruments that can be used with the medical testing device herein. The first—fifth test tube racks are placed in chamber 55C and the sixth test tube rack is placed in chamber 55A.

The 96-plate well resides in chamber 55B for ELISA investigations. The antigen (or antibody) in a buffer solution from the first test tube rack (or containment unit) residing in chamber 55C may be transferred to 96-plate well residing in chamber 55B, via pipette tips in robotic transporter 125. Used pipette tips may be disconnected from robotic transporter 125 and dumped into the sixth test tube rack residing in chamber 55A (see FIGS. 17-20). The 96-plate well residing in chamber 55B may be transferred to an incubator via the pushing mechanism implemented by robotic transporter 131 (see FIGS. 15-17) and adsorbed to the 96-plate well. After incubating for 1 hour at 37 degrees Celsius (C), the 96-plate well may now be pre-coated with the antigen and transported back to chamber 55B via robotic transporter 131.

A complex of an antigen-primary antibody from the second test tube rack in chamber 55A may be transferred to each well of the pre-coated 96-plate well in chamber 55B via robotic transporter 125. At least some of the washing solution from the third test tube rack may be transferred to each well of the pre-coated 96-plate well in chamber 55B to remove unbound antibodies via robotic transporter 131. Enzyme-linked secondary antibody (e.g., peroxidase and alkaline phosphatase) specific to the primary antibody may be added to the 96-plate well in chamber 55B from the fourth test tube rack in chamber 55A. Used tips and/or washings from the 96-plate well in chamber 55B may be dumped into the sixth test tube rack (or containing unit) in chamber 55A.

The 96-plate well, which contains the antigen, the primary antibody, and the secondary antibody, is transferred from chamber 55B to the incubator via robotic transporter 131. After incubating for 1 hour at 37 degrees Celsius (C), the 96-plate well, which contains the antigen, the primary antibody, and the second antibody, is transferred back to chamber 55B. Blood samples or other biological samples collected from clinical subjects/patients, which contain the IL-1 proteins or cytokines, are diluted with buffer solution, and contained in the fourth test tube rack in chamber 55C. A volume of the diluted samples is transferred from the fourth test tube rack in chamber 55C to the 96-plate well, which contains the antigen, the primary antibody, and the secondary antibody, in chamber 55B via robotic transporter 125. The 96-plate well, which contains a combination of the volume of the diluted samples, the antigen, the primary antibody, and the secondary antibody, is transferred from chamber 55B to the incubator via the pushing mechanism implemented by robotic transporter 131.

The 96-plate well, which contains the antigen, the primary antibody, the secondary antibody, and the diluted sample, is transferred from chamber 55B to the incubator via robotic transporter 131. A stop solution is contained in the fifth test tube rack in chamber 55C. A volume of the stop solution is transferred from the fifth test tube rack in chamber 55C to the 96-plate well, which contains the antigen, the primary antibody, the secondary antibody, and the diluted sample in chamber 55B, via robotic transporter 125. The 96-plate well, which contains the antigen, the primary antibody, the secondary antibody, and the diluted sample, may be transferred from chamber 55B to the spectrophotometer via the pushing mechanism implemented by robotic transporter 131. The spectrophotometer can detect the absorption of each well in the 96-plate well at 450 nanometers (nm). The addition of the stop solution may allow some of the sample to be of a particular color. Thus, the ELISA setup may also be amenable for colorimetric analysis and visually identifying positive results (indicative of the presence of IL-1 proteins) or negative result (indicative of the absence of IL-1 proteins).

Quantitative reverse transcriptase, polymerase chain reaction (PCR) assessment of the quantity of IL1 system molecules, or RT-PCR sequencing of blood or oral mucosa swabs for polymorphisms of IL-1RA may be used as complementary tests to assess the IL1 system function.

In chamber 55B, PCR tubes are placed in an optional strip of PCR tube holders stored in a 96-plate well. The PCR tubes from chamber 55B may be transferred to an instrument connected to the medical testing device herein, such as a thermal cycler.

Cell extenders may be added to the whole blood to improve the stability of the mononuclear cells. ELISPOT methods may be used to assess IL-1 system responses of the patients.

For ELISA testing above or other assays, some of the tubes used above are comprised of a Sarstedt Monovette syringe with heparinized growth media pre-filled inside of it together with a stimulant, such as PHA. These tubes may be QFT-Plus testing tubes offered by Quantiferon or T-Cell Xtend® offered by Oxford Immunotec. (U.S. patent application Ser. No. 13/940,758 filed on Jul. 12, 2013, now U.S. Pat. No. 9,005,902) Instead of interferon, IL1 system proteins or other proteins like IL6, IL13, or IL10 may be measured after the stimulation. Other tubes without any additional stimulant are called “Null” tubes and serve as negative controls. A dry well heat block may be used for incubation. In the separated supernatants, the secreted cytokines may be measured using cytokine panel optimized for TruCulture supes called OptiMAP.

Medical Device for Early Diagnosis of Women with Endometriosis: The medical testing devices herein can be used in the diagnosis of endometriosis. Interleukin (IL)-1 is the first identified cytokine and IL-1 system comprises: inducible IL-1β, constitutively expressed IL-1α, IL-1 receptor antagonist (IL-1Ra) and two receptors, IL-1Ra include the biologically active IL-1 receptor type 1(IL-R1), and the decoy IL-1 receptor type 2 (IL-R2). IL-1Ra is an acute phase reactant, similar to c-reactive protein, and it regulates the activity of IL-1β. IL-1Ra binds to IL-1R1 with high affinity and inhibits IL-1α or 1β activity. A soluble form of the IL-1 receptor accessory protein (AcP, sIL-1RAcP) enhances IL-1α and IL-1β binding to IL-1R2 by approximately 100-fold. However, the soluble form of the IL-1 receptor accessory protein does not affect the low binding affinity of IL-1Ra and thereby decreasing IL-1α and β binding to IL-1R1 and reducing inflammation.

TABLE
IL-1 system responses during different phases of
menstrual cycle in female reproductive system

| | Endometrium | Fallopian tubes |
|---|---|---|
| Proliferative phase | IL-1R ↓, IL-1B↓ | IL-1Ra↓ |
| Secretory phase | IL-1R ↑, IL-1B↓ | IL-1Ra↑ |

Interleukin-1α and IL-1β have been shown to upregulate the Cox2 enzyme expression in human myometrial cells ex vivo. Cox2 plays a role in the synthesis of prostaglandins, which lead to myometrial contractility and pain during endometriosis. IL-1 system is activated by multiple extrinsic and intrinsic factors that activate the apoptotic pathways (caspase 1) and inflammasome system, which contribute to the pathogenesis of endometriosis. Toll-like receptor 4 (TLR4) induces the transcription and activation of IL-1 system molecules. Interleukin-1β has been shown to regulate angiogenesis in cancers and vascular health and development of atherosclerosis through NLRP3. IL-1β is involved in both the adhesion and proliferation of endometrial cells and plays role in the progression of endometriosis. Estrogen receptor (ER) β activity is increased in the endometriotic tissues; ERβ interacts with components of the cytoplasmic inflammasome and caspase 1 to increase IL-1B. IL-1B regulates the expression of IL-1Ra. During immune activation, the levels of circulating IL-1Ra and of cellular IL-1R2 determine whether a proinflammatory response will begin, persist, or cease.

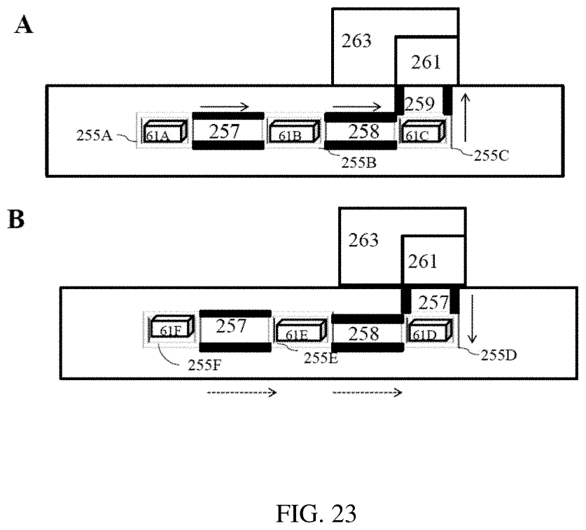

Since IL-1 system is at the center of inflammatory, apoptotic, infectious, endocrine and vascular molecular pathways that lead to the pathogenesis of endometriosis, IL-1Ra is measured by the medical device herein to timely identify women who have endometriosis. By virtue of women with endometriosis being otherwise healthy and mostly experiencing symptoms only during the menstrual cycle, biomarker studies have not shown any difference in the levels of IL-1 system components between women with history of endometriosis and those who do not. Differences in IL-1 system function emerge when the immune system is stressed with a mitogen. The medical device herein is used to perform Experiment 1 and Experiment 2 to identify endometriosis in an expedited and accurate fashion using setup for specialized tube 195 as used for the body for transferring equipment (as described above), plunger system 205, and valve 204. The setup for specialized tube 195, plunger system 205, and valve 204 can be used with the setup of the medical devices herein, as depicted in FIG. 23.

The setup for specialized tube 195 comprises: plunger 150, stopper 155, specialized tube 160, screw cap 165, rubber seal 170, and culturing medium 175. Plunger 150 is a mechanical breakaway plunger which can transform the syringe or transferring equipment for a culture tube. Stopper 155 can prevent injection of culturing medium 175 into a patient. Culturing medium 175 contains agar and LPS to stimulate the growth of IL-proteins. Screw cap 165 and rubber seal 170 can aid in creating a closed system before and after collecting (i.e., drawing) blood, while reducing the risk of biological sample contamination.

Needle system 200 comprises: connection joint 180 to form a supple fit with rubber seal 170, plastic tubing 185 to transfer collected blood from a sample, and needle 190 for making a skin puncture in the patient for collecting blood. Needle system 200 can be connected to the setup for specialized tube 195 to collect blood from a patient. An operator would have to sterilize a section of a patient arm or body prior to collecting blood using needle system 200 and setup for specialized tube 195. The collected blood can be received in specialized tube 160, which allows culturing medium 175 to act upon the collected blood. For example, the clinical integrity of the blood is maintained and can be incubated for up to 48 hours without premature degradation. After disconnecting needle system 200 from setup of specialized tube 195 for a patient, setup of specialized tube 195 for the patient is placed in rack 61A. This process is done for each patient. Rack 61A may have 50 wells and therefore a sample from each of the 50 patients is placed in rack 61A.

Hand 112 of the medical testing device herein can be used to assemble setup for specialized tube 195. For example, each well of a first 96-plate rack contains stimulant and culturing medium 175. Each well of a second 96-plate rack contains an assembly of specialized tube 160, screw cap 165, and rubber seal 170. Hand 112 moves along an x-arm and y-arm to simultaneously uptake the stimulant and culturing medium 175 contents of each respective well of the first 96-plate rack and transfer to a respective well of the second 96-plate rack. In each well of a third 96-plate rack contains an assembly of plunger 150 and stopper 155. Hand 112 moves along an x-arm and y-arm to simultaneously: (i) uptake the assembly of plunger 150 and stopper 155 of each respective well of the third 96-plate rack; and (ii) connect a respective assembly of plunger 150 and stopper 155 to a respective assembly of specialized tube 160, culturing medium 175, screw cap 165, and rubber seal 170. Accordingly, the connection of these assemblies yields setup for specialized tube 195.

Plunger system 205 comprises ridged body 203 which has ridges 202 and plunger 201. Ridged body 203 can makes a supple fit with valve 204. For a biological sample contained within specialized tube 160, the inner walls of setup for specialized tube 195 can make a supple and air tight with valve 204 and plunger system 205 can adjust the height of valve 204. This supports agitation leading to a clean separation of the supernatant phase from cells without centrifugation.

As described above, the robotic transfer units can be used to automate and reduce variability when testing and assessing IL1 protein levels. Referring to FIG. 23, the letter after rack 61 (e.g., A, B, C, etc.) refers to different chambers and times that rack 61 resides in. Setup for specialized tube 195 for each patient is placed in rack 61 to yield rack 61A in chamber 255A. Hand 112 of the robotic transfer unit uses the y-axis arm to latch onto plunger 150 and remove plunger 150 and subsequently using the x-arm to push or move rack 61 along to pathway 257 to yield rack 61B. Chamber 225B for 61B can be used to shake tube 160. The x-arm can then push or move rack 61 along pathway 258 to yield rack 61C in chamber 225C. Chamber 225C for rack 61B can be used to perform inverting steps. The x-arm can push or move rack 61 along pathway 259 into unit 261, which is a receiving region operatively connected to the operating region of unit 263 equipped with, for example, a drying oven. This can be used to grow and modify cells and proteins structures without variability across multiple biological samples. Multistep processes when performed by humans can introduce variability as each biological sample is often handled individually. Thus, multiple biological samples are difficult to or unlikely to be handled uniformly by human operators. In contrast, the medical device herein can handle multistep processes uniformly across multiple biological samples, as exposure and tumbling conditions for racks 61B and 61C are identical across each biological sample.

After being exposed to the dry oven, rack 61 is moved back to a chamber (i.e., chamber 255D) connected to unit 261 along path 257 to yield rack 61D. Rack 61D is subjected to agitation leading to a clean separation of the supernatant phase from cells without centrifugation by having plunger system 205 connected to specialized tube 160 via valve 204. Rack 61 is equipped with plunger system 205 in each well to yield rack 61F in chamber 255F. Hand 112 in the robotic transfer units moves in the y-axis direction to latch onto plunger 201. The position and arrangement of plunger 201 relative to ridged body 203 remains unchanged in chamber 225F. Hand 112 then moves in −y-axis direction to lift each unit of plunger system 205 from the grounded level of rack 61F. In this physically elevated position, hand 112 is gripped onto each unit of plunger system 205 and moves along pathway 257 by the x-arm and is placed directly over rack 61E in chamber 255E. Rack 61E contains valve 204 in each well. Hand 112, which has the gripped units of plunger system 205, moves in the y-axis to attach the gripped of plunger system 205 to a respective unit of valve 204. Hand 112 moves in the −y-axis direction to lift each unit of the gripped plunger system 205 and the respective unit of valve 204. In this physically elevated position, hand 112 moves each unit of the gripped plunger system 205 and the respective unit of valve 204 along pathway 258 by the x-arm and is placed directly over rack 61D in chamber 255D. Hand 112 moves in the y-direction to yield plunger system 205 connected to specialized tube 160 via valve 204.

For each patient, 1 mL of venous blood is drawn into specialized tube 160 containing LPS (e.g., concentration of LPS is 100 ng/mL) of a first unit of setup for specialized tube 195 upon connecting the first unit of setup for specialized tube 195 to a first unit of needle system 200; and 1 mL of venous blood is drawn into specialized tube 160 containing CD3/CD28 (e.g., concentration of CD3/CD28 is 0.2 μg/mL) of a second unit of setup for specialized tube 195 upon connecting the second unit of setup for specialized tube 195 and a second unit of needle system 200. Blood can be drawn for white blood cell counts and differential by using the medical device herein in combination with laboratory techniques known in the art. After 22-24 hours of stimulation of the biological sample on a heating block (e.g., unit 263), as depicted in inset A of FIG. 23, the biological sample is subjected to conditions in inset B of FIG. 23. Thus, for inset B of FIG. 23, cell free supernatant can result for specialized tube 160 containing LPS and the heated and cooled biological sample; and specialized tube containing CD3/CD28 and the heated and biological sample. PHA can also be used a stimulant in specialized tube 160 at the concentration of 20 μg/mL.

For specialized tube 160 containing LPS and the heated and cooled biological sample which has been separated to have cell free supernatant, the IL1 and IL1-Ra levels are divided by the total white blood cell count to yield a first correction value. For specialized tube 160 containing CD3/CD28 and the heated and cooled biological sample which has been separated to have cell free supernatant, the IL1 and IL1-Ra levels are divided by the absolute lymphocyte count as a second correction factor.

Based on the first correction factor and the second correction factor, the patient can be assed for endometriosis. The first correction factor and second correction factor provide normalized IL1 system component expression for cell counts. Additionally, (i) absolute levels of IL-1 and IL-1Ra are compared between women with endometriosis and women without endometriosis; and (ii) per cell production of IL-1 and IL-1Ra are compared between women with endometriosis and women without endometriosis.

Experiment 1: Endometriosis Diagnosis of Women with Pelvic and Menstrual Pain Undergoing Laparoscopy (WUL)

The quantitative level of IL-1 system component production will be measured in women with severe menstrual pain or pelvic pain or dyspareunia for early diagnosis of those with endometriosis. For the assay herein, the blood can be drawn using standard sterile techniques and a catheter into a specialized heparinized tube with the stimulant during the first few days of the menstruation period of the menstrual cycle. The tubes will then be incubated using a dry heat block at 37 C for 22-24 hours, the cell free portion will be separated using a special filtered plunge, the IL-1B and IL-1Ra level in the cell free supernatant will be measured by using multiplex Luminex and the value will be normalized by dividing the value by the total peripheral blood white blood cell count for LPS stimulation or lymphocyte count for the CD3/CD28 stimulation of the patient at the time of the assay. IL-1 function may be calculated as the fold increase over that seen in control treated tubes, or a threshold average diagnostic value of IL-1 system response per ml of blood and/or per cell may be identified for women with endometriosis.

The severity of endometriosis is assessed by using the modified Biberoglu and Behrman scale. The modified Biberoglu and Behrman scale comprises three patient-reported symptoms (dysmenorrhea, dyspareunia, and non-menstrual pelvic pain). Each of these is separately graded on a scale from 0 to 3, with a max of 9 and higher numbers indicating more severe symptoms. The endometriosis induced IL-1 system assay herein can also be correlated with the scores of Biberoglu and Behrman scale comprises three patient-reported symptoms.

Peripheral blood is sent for a white blood cell count with differential. A separate 1 mL of peripheral venous blood is drawn directly into a specialized tube (FIG. 22) using standard sterile techniques. The specialized tube 160 is labeled with the subject ID number and protocol number. Operating region in unit 263 (FIG. 23) is equipped as a dry heating block. The specialized tube is gently inverted to mix the contents and placed in the heating block at 37° C. for 24 hours. At the end of the 24 hours of stimulation, the cell free contents of the specialized tube may be aliquoted and are frozen and stored at −20° C. or lower and shipped frozen to a contracted laboratory to measure IL-1 system components.

Experiment 2: Women Treated with Anakinra (WTA)

The endometriosis induced IL-1 system function assay herein may be used to determine which women may benefit from treatment with recombinant IL-1Ra, Anakinra. sample size of twenty women with laparoscopic diagnosis of endometriosis consent to participate to treatment with IL-1 Antagonist Anakinra (e.g., “Pilot Study of the IL-1 Antagonist Anakinra for the Treatment of Endometriosis Related Symptoms”). A patient within this sample is asked to come to the clinic for blood draw during the first or second day of the menstrual period before the initiation of anakinra or placebo. After obtaining consent form, data will be collected on patient demographics (age, ethnicity, race, weight, height), duration of symptoms, location and severity of symptoms, medications, medical history, surgical history, mental history, history of drug/alcohol/tobacco/nape use, age of menarche, pregnancy history, history of STD, history of birth control use. The results of the laparoscopy, diagnosis, and if present, laparoscopic endometriosis staging are compared to the findings derived from using the medical device herein.

The severity of endometriosis is assessed by using the modified Biberoglu and Behrman scale. The modified Biberoglu and Behrman scale comprises three patient-reported symptoms (dysmenorrhea, dyspareunia, and non-menstrual pelvic pain). Each of these is separately graded on a scale from 0 to 3, with a max of 9 and higher numbers indicating more severe symptoms.

Peripheral blood is sent to a laboratory for white blood cell count with differential. A separate 1 mL of peripheral venous blood is drawn directly into a specialized tube provided using standard sterile techniques. The specialized tube 160 is labeled with the subject. ID number and the protocol number. Operating region in unit 263 is equipped as a dry heating block. The specialized tube is gently inverted to mix the contents and placed in the heating block at 37° C. for 24 hours. At the end of the 24 hours, the contents of the specialized tube are frozen and stored at −20° C. or lower and shipped frozen in dry ice in batches of 50 biological samples to a contracted laboratory.

The same process will be repeated after 6 days of study treatment to assess the impact of the study intervention on the test results. Peripheral blood is sent to the for white blood cell count with differential. A separate 1 mL of peripheral venous blood is drawn directly into a specialized tube provided using standard sterile techniques. The specialized tube 160 is labeled with the subject ID number and the protocol number. Operating region in unit 63 is equipped as a dry heating block. The specialized tube is gently inverted to mix the contents and placed in the heating block at 37° C. for 24 hours. At the end of the 24 hours, the cell free liquid contents of the specialized tube are aliquoted. IL-1 system component expression is measured by using multiplex Luminex technology.

The results of the induced IL-1 system assessment will help the physician determine which patient will respond to the Anakinra treatment.

### Example 3. Immune System Measurements

In the following experiments, 1-Blood may be drawn at a specific time of the day in all patients undergoing RPI test to standardize the comparisons (8-11 am), and 2—The immune responses (cytokine/chemokine levels) post stimulation may be normalized by patient total white blood cell count &/or monocyte count &/or neutrophil count per milliliter of blood.

Uses of ex vivo whole blood or PBMC immune phenotyping in different clinical setting (to screen patients to identify those who have an increased predisposition for the disease, diagnose patients with a disease, identify patients who may respond to a treatment, identify patients who may have a reaction to a treatment or vaccine, identify patients who may have more severe or aggressive disease course)

Experiment 1

NIH US National Library of Medicine Immunophenotyping MeSH description in 2020 is a process of classifying cells of the immune system based on structural and functional differences. Here we describe immune phenotyping as ex vivo stimulation of whole blood to assess the immune responses of a person to specific antigen (s) that has been shown to play a key role in the pathogenesis of the disease

For example one of the main reasons the public refuses to get influenza vaccine is the past experience of severe adverse reaction with body aches and flu like symptoms. Vaccines and influenza vaccination have been shown to differentially induce cytokine release in different people; which may be detected ex vivo. Our technology may help identify people who may have a severe reaction to a vaccine if they were to get immunized.

### REFERENCES

Rapid changes in serum cytokines and chemokines in response to inactivated influenza vaccination. Talaat K R, Halsey N A, Cox A B, Coles C L, Durbin A P, Ramakrishnan A, Bream J H. Influenza Other Respir Viruses. 2018 Mar.; 12(2):202-210. doi: 10.1111/irv.12509. Epub 2018 Jan. 4. PMID: 28991404.
Simon W L, Salk H M, Ovsyannikova I G, Kennedy R B, Poland G A. Cytokine production associated with smallpox vaccine responses. Immunotherapy. 2014; 6:1097-1112.
Maltezou H C, Maragos A, Katerelos P, et al. Influenza vaccination acceptance among health-care workers: a nationwide survey. Vaccine. 2008; 26:1408-1410.
Mangtani P, Breeze E, Stirling S, Hanciles S, Kovats S, Fletcher A. Cross-sectional survey of older peoples' views related to influenza vaccine uptake. BMC Public Health. 2006; 6:249.
Palmore T N, Vandersluis J P, Morris J, et al. A successful mandatory influenza vaccination campaign using an innovative electronic tracking system. Infect Control Hosp Epidemiol. 2009; 30:1137-1142.
Millner V S, Eichold 2nd B H, Franks R D, Johnson G D. Influenza vaccination acceptance and refusal rates among health care personnel. South Med J. 2010; 103:993-998.
Gargano L M, Painter J E, Sales J M, et al. Seasonal and 2009 H1N1 influenza vaccine uptake, predictors of vaccination, and self-reported barriers to vaccination among secondary school teachers and staff. Hum Vaccin. 2011; 7:89-95.

Experiment 2

Ex vivo induced immune responses of whole blood or PBMC may be used to identify patients who may respond to treatment. Thirty-two patients with active rheumatoid arthritis (RA) were treated for up to 24 weeks with weekly intramuscular or subcutaneous injections of methotrexate MTX (15 mg). Active R A was defined by fulfillment of at least 3 of the following 4 criteria: 6 or more joints tender or painful on motion, 3 or more swollen joints, erythrocyte sedimentation rate (ESR) >28 mm/h, and morning stiffness >45 min duration. MTX and dosage of concomitant nonsteroidal anti-inflammatory drugs (NSAID) and steroids (<7.5 mg prednisone/day) were kept constant during the whole study. Clinical assessment was performed before and 6, 12, and 24 weeks after MTX therapy. Laboratory assessment before and during treatment included ESR, routine hematology, erythrocyte folinic acid, serum transaminases, alkaline phosphatase, and creatinine. After 12 weeks of MTX treatment at a constant weekly dosage of 15 mg the patients were divided into 2 groups, responders and nonresponders, according to a composite activity index33. Patients showing improvement of >50% compared to baseline were defined as responders, and patients with improvement <20% or those who deteriorated upon treatment were classified as nonresponders.

Venous blood was drawn from RA patients 24 h after the last intramuscular or subcutaneous injection of MTX, and PBMC were isolated by Ficoll-Hypaque fractionation. The cells were washed 3 times in phosphate buffered saline (PBS) and resuspended in culture medium (10×6 cells/ml). The number of monocytes was determined by differential counting after staining for nonspecific esterase. Monocyte counts in patients' PBMC preparations ranged between 19 and 36% before treatment, and no statistically significant intra or intergroup or intraindividual differences were observed throughout the study. Cells (2×105) in 0.2 ml RPMI 1640 supplemented with 100 IU/ml penicillin/streptomycin (Gibco, Basel, Switzerland) and 1% pasteurized plasma protein solution (5% PPL SRK, Swiss Red Cross) were incubated with or without lipopolysaccharide from E. coli (100 ng/ml; Gibco), phytohemagglutinin (1 μg/ml; Sigma Chemicals, Buchs, Switzerland), IL-1B (10 ng/ml; R&D Systems, London, UK), and with or without MTX (10 ng/ml; AHP AG, Zug, Switzerland) in flat-bottom microtiter plates (Nunc, Roskilde, Denmark) in a humidified atmosphere of 5% CO2 at 37° C. for 48 h. These culture conditions proved to be optimal for assessing IL-10 after LPS and PHA stimulation in PBMC supernatants in prior experiments. Cell-free culture supernatants were collected and stored at −70° C. until use.

IL-10 was determined by a specific human IL-10 ELISA (R&D Systems). The lower detection limit of this assay was 3.9 μg/ml. PBMC of patients with RA showing >50% improvement of the Paulus index after 3 and 6 months of MTX treatment (responders; n=18) exhibited significantly enhanced IL-10 production after in vitro stimulation with LPS, whereas constitutively released IL-10 was below the detection limit of the immunoassay in all patients and controls. In contrast, IL-10 release from LPS stimulated PBMC of RA patients who showed <20% improvement by Paulus index (nonresponders; n=14) or who even deteriorated compared to baseline disease activity was markedly downregulated during MTX treatment in vivo. PHA-induced IL-10 release from PBMC in vitro was not significantly affected by MTX in vivo whether RA patients responded or not to MTX.

### REFERENCES

Enhanced in vitro induced production of interleukin 10 by peripheral blood mononuclear cells in rheumatoid arthritis is associated with clinical response to methotrexate treatment.
M Seitz, M Zwicker and B Wider, http://www.jrheum.org/content/28/3/496

Experiment 3

Ex vivo induced PBMC or whole blood immune responses may be used to identify patients who are going to have a more severe disease course. For example patients with recent-onset rheumatoid arthritis (RA) had decreased ex vivo IL1b production and increased ex vivo IL1Ra production compared with controls. Ex vivo IL1Ra production was shown as an independent predictor of progression of joint damage in recent-onset RA.

Peripheral blood samples were taken at the first visit to the outpatient clinic, before initiation of treatment with DMARDs. In the controls, the peripheral blood samples were taken after a physical examination by a physician to confirm healthy state. Blood samples were collected in pyrogen-free heparinised tubes between 8:00 and 11:00. The samples were cultured within 2 h after collection, with and without (negative control) LPS 10 ng/ml, in 4 ml tubes for 24 h, after which the supernatant was collected and stored at 270° C. IL1b and IL1Ra were measured by ELISA (IL1b: Sanquin, Amsterdam, The Netherlands; IL1Ra: Biosource, Camarillo, Calif., USA) at the same time in all samples in one batch. At the time of sample collection the patients had normal white cell counts. Genotyping was done for polymorphism analyses. The polymorphism CRT at position 511 (rs 16944) in the promoter region of the IL1b gene was typed in all patients and controls with DNA available (n=72 patients; n=61 controls). Primer sequences and PCR conditions were: forward primer 59 GGT AAC AGC ACC TGG TCT TGC-39; reverse primer 59 GCA CAT ACT TTT CTT CAT TCA CTT C-39; PCR cycles: 95° C. for 5 min, followed by 35 cycles of 95° C. for 30 s, 55° C. for 1 min and 72° C. for 30 s, followed by 10 min at 72° C. PCR products were digested with Aval at 37° C. for 90 min and digests were resolved on 2.5% agarose gels. Samples were typed by visual examination of the present size fragments; 10% of all typings were repeated. The error rate was, 0.5%. Radiographs of hands and feet at baseline and after 2 years of follow-up were available for 70 of 76 patients. Radiographic progression was determined (Sharp-van der Heijde score, SHS) by using the mean score of two physicians, who scored the radiographs paired, independently, in random order, blinded for clinical data. Median (interquartile range, IQR) progression was 1.0 (0.0-3.8), mean (SD) 4.7 (11.7), which was comparable with the progression observed in the other patients participating in the BeSt Study (p=0.474). Three groups of patients were defined (tertiles): (1) non-progressive RA, progression score (0; (2) mildly progressive RA, progression score 0.0 and (2; and (3) severely progressive RA, progression score 0.2.

### REFERENCE

Ann Rheum Dis 2007; 66:1033-1037. doi: 10.1136/ard.2006.062463

### OTHER EMBODIMENTS

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing descriptions which do not depart from the spirit or scope of the present inventive discovery. Such modifications are also intended to fall within the scope of the appended claims.

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

What is claimed is:

1. A medical testing device comprising:
a base component comprising a door unit and a plurality of chamber units;
a plurality of walls operatively connected to the base component;
a robotic unit operatively connected to at least one wall of the plurality of walls and a volume transfer unit;
an assay section operatively connected to at least one chamber unit of the plurality of chamber units;
a computer unit operatively connected to the base component; and
a plurality of instruments for measuring characteristics of samples handled by the robotic unit.

2. The medical testing device of claim 1, wherein the base component further comprises a magnetic field creation device, an electric field creation device, a heat generating device, and a spinning device.

3. The medical testing device of claim 1, wherein the plurality of chamber units is operatively connected to sample containing section and waste section.

4. The medical testing device of claim 1, wherein the robotic unit is operatively connected to the volume transfer unit through a support unit.

5. The medical testing device of claim 1, wherein the assay section is a well coated for enzyme-linked immunosorbent assay (ELISA).

6. The medical testing device of claim 5, wherein the well coated for ELISA is operatively connected to a plurality of magnetic agitators.

7. The medical testing device of claim 1, wherein the assay section is a well for housing tubes for polymerase chain reaction (PCR).

8. The medical testing device of claim 1, wherein the plurality of instruments comprises at least one of a thermal cycler, a flow cytometry machine, an incubator, and a photometer.

9. The medical testing device of claim 4, wherein the support unit is a plurality of pipettes.

10. The medical testing device of claim 1, wherein the volume transfer unit is a pipette tip or syringe needle.

11. The medical testing device of claim 1, wherein the measured characteristics comprise protein levels, antibody levels, and cell levels.

12. The medical testing device of claim 6, wherein the plurality of magnetic agitators comprises at least one of a magnet for column-free immunomagnetic separation and bacterial magnetic particles.

13. The medical testing device of claim 1, wherein the computer unit controls parameters for the assay section to perform ELISA, PCR, and multiplex Luminex assay.

14. The medical testing device of claim 11, wherein the protein levels are directed to cytokines.

15. A method for measuring levels of proteins using a medical device acting on specialized tubes, wherein the medical device comprises:
a base component comprising a door unit and a plurality of chamber units;
a plurality of walls operatively connected to the base component;
a robotic unit operatively connected to at least one wall of the plurality of walls and a first volume transfer unit;
an assay section operatively connected to at least one chamber unit of the plurality of chamber units;
a computer unit operatively connected to the base component;
a plurality of instruments for measuring characteristics of samples handled by the robotic unit, wherein the samples contain the proteins; and
wherein the robotic unit moves in a first direction and second direction for uniform handling of at least two samples contained within the specialized tubes.

16. The method of claim 15, wherein uniform handling of the at least two samples containing within the specialized tubes comprises:
stimulating whole blood in the at least two samples within a respective specialized tube, wherein the respective specialized tube is in a negatively pressurized heparinized sterile tube which has a test stimulant or positive control;
sending the at least two samples to an in-tube dry heat block for incubation;
separating cells from supernatant within the at least two samples using a filtered tip plunger; and
using multiplex Luminex assay for quantitatively assessing the supernatant and normalizing the measurement by using white blood cell count or total lymphocyte count before reporting as normal or abnormal.

17. The method of claim 16, wherein the first volume transfer unit and the second volume transfer unit are pipette tips or syringe needles.

18. The method of claim 15, wherein the assay section is a well, wherein the cell is coated for an enzyme-linked immunosorbent assay (ELISA) or multiplex Luminex assay.

19. The method of claim 16, further comprises:
transferring the samples through the robotic unit between the plurality of housing units and the plurality of instruments;
controlling magnetic fields and electric fields through the base component and the computer unit.

20. The method of claim 15, wherein the plurality of instruments comprises at least one of a thermal cycler, a flow cytometry machine, an incubator, and a photometer.
